(19) United States
(12) Patent Application Publication
(10) Pub. No.: US 2003/0219731 A1
(43) Pub. Date: Nov. 27, 2003

(54) Methods for characterizing molecular interactions using affinity capture tandem mass spectrometry

(75) Inventors: Scot R. Weinberger, Montara, CA (US); Tina Morris, Rockville, MD (US)

Correspondence Address: FISH & NEAVE, 1251 AVENUE OF THE AMERICAS, 50TH FLOOR, NEW YORK, NY, 10020-1105, US

(73) Assignees: CIPHERGEN BIOSYSTEMS, INC., Fremont, CA; HUMAN GENOME SCIENCES, INC., Rockville, MD

(21) Appl. No.: 10/406,023

(22) Filed: Apr. 1, 2003

Related U.S. Application Data
(63) Continuation of application No. PCT/US00/28261, filed on Oct. 11, 2000.

(51) Int. Cl. C12Q 1/70; C12Q 1/68; G01N 33/53; G01N 33/543

(52) U.S. Cl. 435/5; 435/6; 435/7.1; 435/7.5; 436/518

(57) ABSTRACT

The invention provides an analytical instrument comprising an affinity capture probe interface, a laser desorption ionization source, and a tandem mass spectrometer. Also presented are new methods for protein discovery and identification and for characterization of molecular interactions that utilize the instrument of the present invention.

### FIELD OF THE INVENTION

This invention is in the field of chemical and biochemical analysis, and relates particularly to apparatus and methods for improved identification and characterization of analytes and of affinity interactions between analytes by tandem mass spectrometry.

### BACKGROUND OF THE INVENTION

The advent of electrospray ionization (ESI) and matrix-assisted laser desorption/ionization (MALDI) techniques, coupled with improved performance and lower cost of mass analyzers, has in the past decade allowed mass spectrometry (MS) to take a place among standard analytical tools in the study of biologically relevant macromolecules, including proteins purified from complex biological systems.

For example, in a technique known as peptide mass fingerprinting, mass spectrometry is used to identify proteins purified from biological samples. Identification is effected by matching the mass spectrum of proteolytic fragments of the purified protein with masses predicted from primary sequences prior-accessioned into a database. Roepstorff, The Analyst 117:299-303 (1992); Pappin et al., Curr. Biol. 3(6):327-332 (1993); Mann et al., Biol. Mass Spectrom. 22:338-345 (1993); Yates et al., Anal. Biochem. 213:397-408 (1993); Henzel et al., Proc. Natl. Acad. Sci. USA 90:5011-5015 (1993); James et al., Biochem. Biophys. Res. Commun. 195:58-64 (1993).

Similar database-mining approaches have been developed that use fragment mass spectra obtained from collision induced dissociation (CID) or MALDI post-source decay (PSD) to identify purified proteins. Eng et al., J. Am. Soc. Mass. Spectrom. 5:976-989 (1994)); Griffin et al., Rapid Commun. Mass Spectrom. 9:1546-1551 (1995); Yates et al., U.S. Pat. Nos. 5,538,897 and 6,017,693; Mann et al., Anal. Chem. 66:4390-4399 (1994).

Mass spectrometric techniques have also been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

Software resources that facilitate interpretation of protein mass spectra and mining of public domain sequence databases are now readily accessible on the internet to facilitate protein identification. Among these are Protein Prospector (http://prospector.ucsf/edu), PROWL (http://prowl.rockefeller.edu), and the Mascot Search Engine (Matrix Science Ltd., London, UK, www.matrixscience.com).

Although highly accurate mass assignment provides useful information—facilitating identification of purified protein by the above-described techniques, for example—such information is nonetheless limited. Significant additional analytical power would be unleashed by combining MS analysis with enzymatic and/or chemical modification of target proteins, enabling the elucidation of structural components, post-translational modifications, and furthering protein identification.

Furthermore, complex biological materials—such as blood, sera, plasma, lymph, interstitial fluid, urine, exudates, whole cells, cell lysates and cellular secretion products—typically contain hundreds of biological molecules, plus organic and inorganic salts, which precludes direct mass spectrometry analysis. Thus, significant sample preparation and purification steps are typically necessary prior to MS investigation.

Classical methods of sample purification, such as liquid chromatography (ion exchange, size exclusion, affinity, and reverse phase chromatography), membrane dialysis, centrifugation, immunoprecipitation, and electrophoresis, typically demand a large quantity of starting sample. Even when such quantities of sample are available, minor components tend to become lost in these purification processes, which suffer from analyte loss due to non-specific binding and dilution effects. The methods are also often quite labor intensive.

Thus, there is a clear need for methods and apparatus that facilitate mass spectrometric detection of both major and minor proteins present in heterogeneous samples without requiring extensive prior fluid phase purification. There is further need for an MS platform that allows not only facile sample purification, but also permits serial and parallel sample modification approaches prior to mass spectrometric analysis.

These needs have been met, in part, by the development of affinity capture laser desorption ionization approaches. Hutchens et al., Rapid Commun. Mass Spectrom. 7: 576-580 (1993); U.S. Pat. Nos. 5,719,060, 5,894,063, 6,020,208, and 6,027,942. This new strategy for MS analysis of macromolecules uses novel laser desorption ionization probes that have an affinity reagent on at least one surface. The affinity reagent adsorbs desired analytes from heterogeneous samples, concentrating them on the probe surface in a form suitable for subsequent laser desorption ionization. The coupling of adsorption and desorption of analyte obviates off-line purification approaches, permitting analysis of smaller initial samples and further facilitating sample modification approaches directly on the probe surface prior to mass spectrometric analysis.

The affinity capture laser desorption ionization approach has allowed mass spectrometry to be adapted to numerous classic bioanalytical assay formats, including immunoassay, Nelson et al., Anal. Chem. 67: 1153-1158 (1995), and affinity chromatography, Brockman et al., Anal. Chem. 67: 4581-4585 (1995). The affinity capture laser desorption ionization approach has been applied not only to the study of peptides and proteins, Hutchens et al., Rapid Commun. Mass Spectrom. 7:576-580 (1993); Mouradian et al., J. Amer. Chem. Soc. 118: 8639-8645 (1996); Nelson et al., Rapid Commun. Mass. Spectrom. 9: 1380-1385 (1995); Nelson et al., J. Molec. Recognition 12: 77-93 (1999).; Brockman et al., J. Mass Spectrom. 33: 1141-1147 (1998); Yip et al., J. Biol. Chem. 271: 32825-33 (1996), but also to oligonucleotides, Jurinke et al., Anal. Chem. 69:904-910 (1997); Tang et al., Nucl. Acids Res. 23: 3126-3131 (1995); Liu et al., Anal. Chem. 67: 3482-90 (1995), bacteria, Bundy et al., Anal. Chem. 71: 1460-1463 (1999), and small molecules, Wei et al., Nature 399:243-246 (1999). At the commercial level, affinity capture laser desorption ionization is embodied in Ciphergen's ProteinChip® Systems (Ciphergen Biosystems, Inc. Fremont, Calif., USA).

Although the affinity capture laser desorption ionization technique has solved significant problems in the art, difficulties remain.

When this approach is applied to capture proteins from biological samples, it is common to see about one picomole of total protein captured and available for subsequent analysis. Typically, affinity capture on chromatographic surface biochips does not result in complete purification. Additionally, the digestion efficiency seen for solid phase extracted samples, as compared to digests performed in free solution or the denaturing environment of 2-D gels, is poor. Thus, if about 50% were the protein of interest, and one were successful in digesting about 10% of this protein, at best only about 50 femtomole of some peptides would be available for detection.

Using virtual tryptic digests of bovine fetuin in database mining experiments, it has been demonstrated, for example, that even with an extreme accuracy of 1.0 ppm (a level not currently achievable by most MS techniques), a poor confidence protein ID match is achieved with a single peptide mass when searching against this complex, eukaryotic genome. For two peptides, low confidence results are achieved as well. Only after three peptides are submitted are confident results returned for mass assignments of less than 300 ppm error. In this case, most devices would require internal standard calibration. However, with five or more peptides, no further confidence is afforded with mass accuracies that are better than 1000 ppm error.

Furthermore, when multiple proteins are simultaneously digested, a heterogeneous peptide pool is created and successful database mining requires not only extreme accuracy, but in many instances primary sequence information. Tandem MS/MS approaches have demonstrated significant utility in providing primary sequence information. Biemann et al., Acc. Chem. Res. 27: 370-378 (1994); Spengler et al., Rapid Commun. Mass Spectrom. 1991, 5:198-202 (1991); Spengler et al., Rapid Commun. Mass Spectrom. 6:105-108 (1992); Yates et al., Anal. Chem. 67:1426-1436 (1995); Kaufman et al., Rapid Commun. Mass. Spectrom. 7:902-910 (1993); Kaufman et al., Intern. J. Mass Spectrom. Ion Processes 131:355-385 (1994).

Until recently, however, the only MS/MS approach available for laser desorption based analyses was post source decay analysis (PSD). While PSD is capable of providing reasonable sequence information for picomole levels of peptides, the overall efficiency of this fragmentation process is low; when combined with the poor mass accuracy and sensitivity often demonstrated during this approach, its applicability to analysis of low abundance proteins often found on affinity capture laser desorption ionization probes has been greatly limited. Recently, a laser desorption ionization quadrupole time-of-flight mass spectrometer (LDI Qq-TOF) has been developed that is capable of performing collision induced dissociation (CID) MS/MS analysis. Krutchinksy et al., Rapid Commun. Mass Spectrom. 12: 508-518 (1998).

There is, therefore, a need for apparatus and methods that would increase the sensitivity and mass accuracy of affinity capture laser desorption mass spectrometry. There is a need for methods and apparatus that would increase on-probe digestion efficiency and that would permit peptides generated by digest of inhomogeneous mixtures of proteins readily to be resolved. There is a need for apparatus and methods that would increase the efficiency of affinity capture laser desorption tandem mass spectrometric analysis.

### SUMMARY OF THE INVENTION

It is an object of the present invention to provide apparatus and methods that increase the sensitivity, mass accuracy, mass resolution of existing affinity capture laser desorption ionization mass spectrometer analyses and to add ms/ms capability. It is a further object of the present invention to provide methods of biomolecule analysis that exploit these improved analytical capabilities.

The present invention meets these and other objects and needs in the art by providing, in a first aspect, an analytical instrument.

The analytical instrument of the present invention comprises a laser desorption ionization source, an affinity capture probe interface, and a tandem mass spectrometer, in which the affinity capture probe interface is capable of engaging an affinity capture probe and positioning the probe so that it can be interrogated by the laser desorption source while in communication with the tandem mass spectrometer, thus permitting ions desorbed from the probe to enter the mass spectrometer.

Typically, the laser desorption ionization source comprises a laser excitation source and a laser optical train; the laser optical train functions to transmit excited photons from the laser excitation source to the probe interface. In such embodiments, the laser optical train typically delivers about 20-1000 microjoules of energy per square millimeter of interrogated probe surface.

The laser excitation source is selected from the group consisting of a continuous laser and a pulsed laser, and in various embodiments is selected from the group consisting of a nitrogen laser, a Nd:YAG laser, an erbium:YAG laser, and a CO2 laser. In a presently preferred embodiment, the laser excitation source is a pulsed nitrogen laser.

In one set of embodiments, the laser optical train comprises optical components selected from the group consisting of lenses, mirrors, prisms, attenuators, and beam splitters.

In an alternative set of embodiments, the laser optical train comprises an optical fiber having an input end and an output end, and the laser excitation source is coupled to said optical fiber input end.

In some of the optical fiber laser optical train embodiments, the laser optical train further comprises an optical attenuator. The attenuator can be positioned between the laser excitation source and the input end of the optical fiber, can serve to couple the laser excitation source to the input end of the optical fiber, or can be positioned between the optical fiber output end and the probe.

In certain of the optical fiber optical train embodiments, the optical fiber output end has a maximum diameter between about 200-400 μm and the input end has a diameter of between about 400 to 1200 μm.

The analytical instrument can also include probe viewing optics, to permit the probe to be visualized after its engagement in the probe interface.

In certain embodiments, the laser optical train can include a laser coupler that couples the laser excitation source to the optical fiber input end. As noted above, the coupler can serve as an optical attenuator. In other embodiments, the coupler can serve to promote visualization of the probe after its engagement in the probe interface.

In certain of these latter embodiments, either the coupler or the fiber is bifurcated and splits off a fraction of energy from said laser excitation source. Alternatively, such bifurcation can allow introduction of visible light to illuminate the desorption locus.

Where visualization optics are included in the optical train, or where a fiber-containing laser optical train includes a bifurcation or trifurcation, the analytical instrument can further comprise a CCD camera positioned to detect light reflected from said probe.

In typical embodiments, the affinity capture probe interface comprises a probe holder which is capable of reversibly engaging the affinity capture probe. The interface also typically comprises a probe introduction port which is itself capable of reversibly engaging the probe holder.

In typical embodiments, the probe interface further comprises a probe position actuator assembly and an interface ion collection system. When the probe holder is engaged in the introduction port, it is placed in contact with the probe position actuator; the probe position actuator, in turn, is capable of movably positioning the probe holder (typically with its engaged probe) with respect both to the laser ionization source (typically, with respect to the laser optical train) and to the ion collection system. In typical embodiments, the actuator is capable of translationally and rotationally positioning said probe holder.

The probe interface typically also comprises a vacuum evacuation system coupled to the probe introduction port, which allows the probe to be interrogated by the laser desorption ionization source at subatmospheric pressures.

The analytical instrument of the present invention comprises a tandem mass spectrometer which, in various embodiments, is selected from the group consisting of a QqTOF MS, an ion trap MS, an ion trap TOF MS, a TOF-TOF MS, and a Fourier transform ion cyclotron resonance MS. Presently preferred for use in the analytical instrument of the present invention is a QqTOF MS.

In preferred embodiments, the tandem mass spectrometer is a QqTOF MS and the laser excitation source is a pulsed nitrogen laser, laser fluence at the probe is about 2 to 4 times the minimum desorption threshold, and the tandem mass spectrometer has an external standard mass accuracy of about 20-50 ppm.

The analytical instrument of the present invention is designed to engage an affinity capture laser desorption ionization probe. Accordingly, any of the above-described embodiments can include an affinity capture probe engaged in the affinity capture probe interface.

The affinity capture probe in these embodiments will typically have at least one sample adsorption surface positioned in interrogatable relationship to the laser source, the sample adsorption surface selected from the group consisting of chromatographic adsorption surfaces and biomolecule affinity surfaces. Typically, such chromatographic adsorption surface is selected from the group consisting of reverse phase, anion exchange, cation exchange, immobilized metal affinity capture and mixed-mode surfaces and the biomolecule of the biomolecule affinity surfaces is selected from the group consisting of antibodies, receptors, nucleic acids, lectins, enzymes, biotin, avidin, streptavidin, Staph protein A and Staph protein G.

The affinity capture laser desorption ionization probe can have a plurality of separately addressable sample adsorption surfaces that can be positioned in interrogatable relationship to the laser source and can include at least two different such adsorption surfaces.

In other embodiments, the analytical instrument of the present invention includes a a digital computer interfaced with a detector of the tandem mass spectrometer. In some embodiments, the instrument can also further include a software program executable by the digital computer, either local to the computer or communicably accessible to the computer. The software program in such embodiments can be capable of controlling the laser desorption ionization source, or of controlling at least one aspect of data acquisition by the tandem mass spectrometer, or of performing at least one analytical routine on data acquired by said tandem mass spectrometer, or any subset of these functions.

In another aspect, the invention provides a method for analyzing at least one test protein.

The method comprises (a) capturing the test protein or proteins on an affinity capture protein biochip, (b) generating protein cleavage products of the test protein(s) on the protein biochip using a proteolytic agent; and (c) analyzing at least one protein cleavage product with a tandem mass spectrometer. In these embodiments of this aspect, the analyzin step comprises (i) desorbing the protein cleavage products from the protein biochip into gas phase to generate corresponding parent ion peptides, (ii) selecting a parent ion peptide for subsequent fragmentation with a first mass spectrometer, (iii) fragmenting the selected parent ion peptide under selected fragmentation conditions in the gas phase to produce product ion fragments and (iv) generating a mass spectrum of the product ion fragments. In this fashion, the mass spectrum provides an analysis of the test proteins.

In certain embodiments of this aspect of the invention, the method further includes an additional step (d), determining at least one protein identity candidate for a test protein by submitting the mass spectrum to a protein database mining protocol which identifies at least one protein identity candidate for the test protein in the database based on a measure of closeness-of-fit between the mass spectrum and theoretical mass spectra of proteins in the database.

In particular of these embodiments, step (d) further comprises submitting the mass of the test protein and the species of origin of the test protein to the protocol.

In other embodiments, the method further comprises (e) comparing the identity candidate to the test protein by: (i) generating a mass spectrum of the protein cleavage products of (b); (ii) submitting the mass spectrum of the protein cleavage products to a computer protocol that determines a measure of closeness-of-fit between the theoretical mass spectrum of cleavage products of the identity candidate predicted to be generated by using the proteolytic agent, and the mass spectrum of the protein cleavage products, whereby the measure indicates protein cleavage products on the protein biochip that correspond to the test protein.

Yet other embodiments of the method include the further steps of (f), repeating step (c) wherein the selected parent ion peptide does not correspond to a protein cleavage product predicted from the identity candidate; and then (g) repeating (d) for the selected parent ion peptide of (f).

In this aspect of the invention, the test protein can be a protein that is differentially expressed between first and second biological samples. In some of these embodiments, the first and second biological samples are derived from normal and pathological sources.

In a third aspect, the invention provides a method of characterizing binding interactions between a first and second molecular binding partner.

In this aspect, the method comprises binding a second binding partner to a first binding partner, where the said first binding partner is immobilized to a surface of a laser desorption ionization probe; fragmenting the second binding partner; and then detecting at least one of the fragments by a tandem mass spectrometer measurement, whereby the mass spectrum of the detected fragments characterizes the binding interactions.

In certain embodiments of this aspect of the invention, the first binding partner is first immobilized to a surface of an affinity capture probe before the second binding partner is bound to the first binding partner.

Such immobilizing can be by direct binding of the first partner to the affinity capture probe, such as a covalent bonding. Typical covalent bonding embodiments include covalent bonding between an amine of the first binding partner and a carbonyldiimidazole moiety of said probe surface and between an amino or thiol group of said first binding partner and an epoxy group of the probe surface.

The immobilizing can also be by direct noncovalent bonding, such as a coordinate or dative bonding between the first binding partner and a metal, such as gold or platinum, of the probe surface. The immobilizing can also be by interaction of the first binding partner to a chromatographic adsorption surface selected from the group consisting of reverse phase, anion exchange, cation exchange, immobilized metal affinity capture and mixed-mode surfaces.

Alternatively, the immmobilizing can be indirect, which can be covalent, albeit indirect. In certain of these latter embodiments, the first binding partner can be immobilized by covalent bonding through a linker, such as a cleavable linker. Indirect immobilization can also be noncovalent, such as immobilization to the probe via a biotin/avidin, biotin/streptavidin interaction.

In this aspect of the invention, the first molecular binding partner can be selected from the group consisting of protein, nucleic acid, carbohydrate, and lipid. Typically, the first binding partner will be a protein, which can be a naturally occurring protein from an organism selected from the group consisting of multicellular eukaryote, single cell eukaryote, prokaryote, and virus, or can be a nonnaturally occurring protein, such as a recombinant fusion protein.

In embodiments in which the first binding partner is a protein, the protein can be selected from the group consisting of antibody, receptor, transcription factor, cytoskeletal protein, cell cycle protein, and ribosomal protein, among others.

Binding of the second binding partner to the immobilized first binding partner is, in typical embodiments, effected by contacting the first binding partner with a biologic sample; the sample can be a fluid selected from the group consisting of blood, lymph, urine, cerebrospinal fluid, synovial fluid, milk, saliva, vitreous humor, aqueous humor, mucus and semen, or a cell lysate, or some sample in another form.

In various embodiments, including embodiments in which the first binding partner is a protein, the second binding partner can be a protein. Alternatively, the second binding partner can be a compound present in a combinatorial library, where binding of the second binding partner to the first binding partner is effected by contacting the first binding partner with an aliquot of a chemically synthesized combinatorial library. In yet other alternatives, the second binding partner can be a component of biologically displayed combinatorial library, such as a phage-displayed library.

In certain typical embodiments, fragmenting is effected by contacting the second binding partner with an enzyme; where the second binding partner is a protein, the enzyme is typically a specific endoprotease, such as trypsin, Glu-C (V8) protease, endoproteinase Arg-C (serine protease), endoproteinase Arg-C (cysteine protease), Asn-N protease, and Lys-C protease. Alternatively, fragmenting can be effected by contacting said second binding partner with a liquid phase chemical, such as CNBr.

In some embodiments, the method further comprises, after binding of the second binding partner to said first binding partner, and before fragmenting the second binding partner, of denaturing the second binding partner.

In various embodiments, the method further comprises the step, after fragmenting the second binding partner, of washing the probe with a first eluant, and, at times, a second eluant, the second eluant differing from the first eluant in at least one elution characteristic, such as pH, ionic strength, detergent strength, and hydrophobicity.

In typical embodiments, the method further comprises, after fragmenting and before detecting the fragments of the second binding partner, the step of applying energy absorbing molecules to the probe. In preferred embodiments, the probe is then engaged in the affinity capture probe interface of the analytical instrument of the present invention, and fragments of the second binding partner ionized and desorbed from the probe using the instrument's laser source.

The instrument can be used to make several types of useful measurements in this method, including a measurement of all ion masses, a measurement of masses of a subset of fragments, and a single ion monitoring measurement.

Usefully, embodiments of the method include the step, after mass spectrometric measurement of fragments of the second binding partner, of comparing the fragment measurements with those predicted by applying cleavage rules of the fragmenting enzyme to the primary amino acid sequence of the second binding partner, whereby such comparison characterizes the intermolecular interactions.

If the identity of the second binding partner is not known, the method can further comprise, before such comparison, identifying the second binding partner through ms/ms analysis. such MS/MS analysis can include the steps of mass spectrometrically selecting a first fragment of the second binding partner; dissociating the second binding partner first fragment in the gas phase; measuring the fragment spectrum of the second binding partner first fragment, and then comparing the fragment spectrum to fragment spectra predicted from amino acid sequence data prior-accessioned in a database. The amino acid sequence data can be selected from the group consisting of empiric and predicted data, and the dissociating, in typically embodiments, is collision induced dissociation.

In some embodiments of the method, the first binding partner is selected from the group consisting of an antibody, a T cell receptor, and an MHC molecule. In other embodiments, the first binding partner is a receptor and the second binding partner is selected from the group consisting of an agonist of the receptor, a partial agonist of the receptor, an antagonist of said receptor, and a partial antagonist of said receptor. In other embodiments, the first binding partner is a glycoprotein receptor and the second binding partner is a lectin.

In a fourth aspect, the invention provides a method of detecting an analyte, the method comprising engaging a affinity capture probe in the affinity capture probe interface of the analytical instrument of the present invention, the affinity capture probe having an analyte bound thereto; desorbing and ionizing the analyte or fragments thereof from the probe using the instrument's laser source; and then detecting the analyte by a tandem mass spectrometer measurement on the desorbed ions.

In this aspect, the method can further comprise, after the desorbing and ionizing step and before detecting, effecting collision induced dissociation of said desorbed ions. Before such dissociation, in some embodiments a subset of ions can be selected for collisional dissociation.

In other embodiments, the antecedent step can be performed of adsorbing analyte to the probe, and in yet other embodiments, a step can be performed after adsorbing analyte and prior to engaging said probe in said probe interface, of adherently contacting said probe and said analyte with energy absorbing molecules.

### BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIG. 1 schematizes an embodiment of the analytical instrument of the present invention;

FIG. 2 shows in greater detail the elements of an orthogonal QqTOF tandem mass spectrometer preferred for use in the analytical instrument of the present invention;

FIG. 3 displays the seminal fluid protein profiles of a single BPH and prostate cancer patient;

FIG. 4 shows results of on-probe isolation of one of the upregulated proteins detectable in FIG. 3;

FIG. 5 shows peptides detected by a single phase of MS analysis after the enriched biomarker candidate was exposed to in situ digestion using trypsin;

FIG. 6 shows LDI Qq-TOF MS analysis of the same purified protein peptides on the analytical device of the present invention; and

FIG. 7 shows MS/MS results from the analytical device of the present invention of a selected doubly charged ion of the enriched biomarker candidate.

### DETAILED DESCRIPTION OF THE INVENTION

I. Definitions

As used herein, the terms set forth with particularity below have the following definitions. If not otherwise defined, all terms used herein have the meaning commonly understood by a person skilled in the arts to which this invention belongs.

“Analyte” refers to any component of a sample that is desired to be detected. The term can refer to a single component or a plurality of components in the sample.

“Probe” refers to a device that, when positionally engaged in interrogatable relationship to a laser desorption ionization source and in concurrent communication at atmospheric or subatmospheric pressure with a gas phase ion spectrometer, can be used to introduce ions derived from an analyte into the spectrometer. As used herein, the “probe” is typically reversibly engageable by a probe interface.

“Affinity capture probe” refers to a probe that binds analyte through an interaction that is sufficient to permit the probe to extract and concentrate the analyte from an inhomogeneous mixture. Concentration to purity is not required. The binding interaction is typically mediated by adsorption of analyte to an adsorption surface of the probe. The term ProteinChip® Array refers to affinity capture probes that are commercially available from Ciphergen Biosystems, Inc., Fremont, Calif., for use in the present invention.

“Adsorption” refers to detectable noncovalent binding of an analyte to an adsorbent.

“Adsorbent” refers to any material capable of adsorbing an analyte. The term “adsorbent” is used herein to refer both to a single material (“monoplex adsorbent”) (e.g., a compound or a functional group) and to a plurality of different materials (“multiplex adsorbent”). The adsorbent materials in a multiplex adsorbent are referred to as “adsorbent species.” For example, a laser-addressable adsorption surface on a probe substrate can comprise a multiplex adsorbent characterized by many different adsorbent species (e.g., anion exchange materials, metal chelators, or antibodies) having different binding characteristics.

“Adsorption surface” refers to a surface having an adsorbent.

“Chromatographic adsorption surface” refers to a surface having an adsorbent capable of chromatographic discrimination among or separation of analytes. The phrase thus includes surfaces having anion exchange moieties, cation exchange moieties, reverse phase moieties, metal affinity capture moieties, and mixed-mode adsorbents, as such terms are understood in the chromatographic arts.

“Biomolecule affinity surface” refers to a surface having an adsorbent comprising biomolecules capable of specific binding.

“Specific binding” refers to the ability of two molecular species concurrently present in a heterogeneous (inhomogeneous) sample to bind to one another preferentially over binding to other molecular species in the sample. Typically, a specific binding interaction will discriminate over adventitious binding interactions in the reaction by at least two-fold, more typically more than 10- to 100-fold. When used to detect analyte, specific binding is sufficiently discriminatory when determinative of the presence of the analyte in a heterogeneous (inhomogeneous) sample. Typically, the affinity or avidity of a specific binding reaction is least about 10⁻⁷ M, with specific binding reactions of greater specificity typically having affinity or avidity of at least 10⁻⁸ M to at least about 10⁻⁹ M.

“Energy absorbing molecules” and the equivalent acronym “EAM” refer to molecules that are capable, when adhered to a probe, of absorbing energy from a laser desorption ionization source and thereafter contributing to the desorption and ionization of analyte in contact therewith. The phrase includes all molecules so called in U.S. Pat. Nos. 5,719,060, 5,894,063, 6,020,208, and 6,027,942, the disclosures of which are incorporated herein by reference in their entireties. The phrase explicitly includes cinnamic acid derivatives, sinapinic acid (“SPA”), cyano hydroxy cinnamic acid (“CHCA”) and dihydroxybenzoic acid.

“Tandem mass spectrometer” refers to any gas phase ion spectrometer that is capable of performing two successive stages m/z-based discrimination of ions in an ion mixture. The phrase includes spectrometers having two mass analyzers as well as those having a single mass analyzer that are capable of selective acquisition or retention of ions prior to mass analysis. The phrase thus explicitly includes QqTOF mass spectrometers, ion trap mass spectrometers, ion trap-TOF mass spectrometers, TOF-TOF mass spectrometers, and Fourier transform ion cyclotron resonance mass spectrometers.

“Eluant” refers to an agent, typically a solution, that is used to affect or modify adsorption of an analyte to an adsorbent of an adsorption surface. Eluants also are referred to herein as “selectivity threshold modifiers.”

“Elution characteristic” refers to a physical or chemical characteristic of an eluant that contributes to its ability to affect or modify adsorption of an analyte to an adsorbent of an adsorption surface. Two eluants have different elution characteristics if, when put in contact with an analyte and adsorbent, the degree of affinity of the analyte for the adsorbent differs. Elution characteristics include, for example, pH, ionic strength, degree of chaotropism, detergent strength, and temperature.

“Biologic sample” and “biological sample” identically refer to a sample derived from at least a portion of an organism capable of replication. As used herein, a biologic sample can be derived from any of the known taxonomic kingdoms, including virus, prokaryote, single celled eukaryote and multicellular eukaryote. The biologic sample can derive from the entirety of the organism or a portion thereof, including from a cultured portion thereof. Biologic samples can be in any physical form appropriate to the context, including homogenate, subcellular fractionate, lysate and fluid.

“Biomolecule” refers to a molecule that can be found in, but need not necessarily have been derived from, a biologic sample.

“Organic biomolecule” refers to an organic molecule that can be found in, but need not necessarily have been derived from, a biologic sample, such as steroids, amino acids, nucleotides, sugars, polypeptides, polynucleotides, complex carbohydrates and lipids.

“Small organic molecule” refers to organic molecules of a size comparable to those organic molecules generally used in pharmaceuticals. The term excludes organic biopolymers (e.g., proteins, nucleic acids, etc.). Small organic molecules as used herein typically range in size up to about 5000 Da, up to about 2500 Da, up to about 2000 Da, or up to about 1000 Da.

“Biopolymer” refers to a polymer that can be found in, but need not necessarily have been derived from, a biologic sample, such as polypeptides, polynucleotides, polysaccharides and polyglycerides (e.g., di- or tri-glycerides).

“Fragment” refers to the products of the chemical, enzymatic, or physical breakdown of an analyte. Fragments may be in a neutral or ionic state.

The terms “polypeptide”, “peptide”, and “protein” are used interchangeably herein to refer to a naturally-occurring or synthetic polymer comprising amino acid monomers (residues), where amino acid monomer here includes naturally-occurring amino acids, naturally-occurring amino acid structural variants, and synthetic non-naturally occurring analogs that are capable of participating in peptide bonds. Polypeptides can be modified, e.g., by the addition of carbohydrate residues to form glycoproteins. The terms “polypeptide,” “peptide” and “protein” include glycoproteins as well as non-glycoproteins.

“Polynucleotide” and “nucleic acid” equivalently refer to a naturally-occurring or synthetic polymer comprising nucleotide monomers (bases). Polynucleotides include naturally-occurring nucleic acids, such as deoxyribonucleic acid (“DNA”) and ribonucleic acid (“RNA”), as well as nucleic acid analogs. Nucleic acid analogs include those which include non-naturally occurring bases, and those in which nucleotide monomers are linked other than by the naturally-occurring phosphodiester bond. Nucleotide analogs include, for example and without limitation, phosphorothioates, phosphorodithioates, phosphorotriesters, phosphoramidates, boranophosphates, methylphosphonates, chiral-methyl phosphonates, 2-O-methyl ribonucleotides, peptide-nucleic acids (PNAs), and the like.

As used herein, “molecular binding partners”—and equivalently, “specific binding partners”—refer to pairs of molecules, typically pairs of biomolecules, that exhibit specific binding. Nonlimiting examples are receptor and ligand, antibody and antigen, biotin and avidin, and biotin and streptavidin.

“Receptor” refers to a molecule, typically a macromolecule, that can be found in, but need not necessarily have been derived from, a biologic sample, and that can participate in specific binding with a ligand. The term further includes fragments and derivatives that remain capable of specific ligand binding.

“Ligand” refers to any compound that can participate in specific binding with a designated receptor or antibody.

“Antibody” refers to a polypeptide substantially encoded by at least one immunoglobulin gene or fragments of at least one immunoglobulin gene, that can participate in specific binding with a ligand. The term includes naturally-occurring forms, as well as fragments and derivatives. Fragments within the scope of the term as used herein include those produced by digestion with various peptidases, such as Fab, Fab′ and F(ab)′2 fragments, those produced by chemical dissociation, by chemical cleavage, and recombinantly, so long as the fragment remains capable of specific binding to a target molecule. Typical recombinant fragments, as are produced, e.g., by phage display, include single chain Fab and scFv (“single chain variable region”) fragments. Derivatives within the scope of the term include antibodies (or fragments thereof) that have been modified in sequence, but remain capable of specific binding to a target molecule, including interspecies chimeric and humanized antibodies. As used herein, antibodies can be produced by any known technique, including harvest from cell culture of native B lymphocytes, hybridomas, recombinant expression systems, by phage display, or the like.

“Antigen” refers to a ligand that can be bound by an antibody. An antigen need not be immunogenic. The portions of the antigen that make contact with the antibody are denominated “epitopes”.

“Fluence” refers to the energy delivered per unit area of interrogated image.

II. Affinity Capture Probe Tandem Mass Spectrometer

In a first aspect, the present invention provides an analytical instrument that combines the advantages of affinity capture laser desorption ionization sample introduction with the advantages of high accuracy, high mass resolution, tandem mass spectrometers. The combination provides significant advantages over existing devices for performing known techniques. Furthermore, the new instrument makes possible new methods of protein discovery and makes possible new methods of identifying and characterizing molecular interactions between and among specific binding partners that are more at once more efficient and more sensitive than existing approaches. The instrument will first briefly be described as a whole; thereafter, features of the affinity capture probe interface will be described in greater detail.

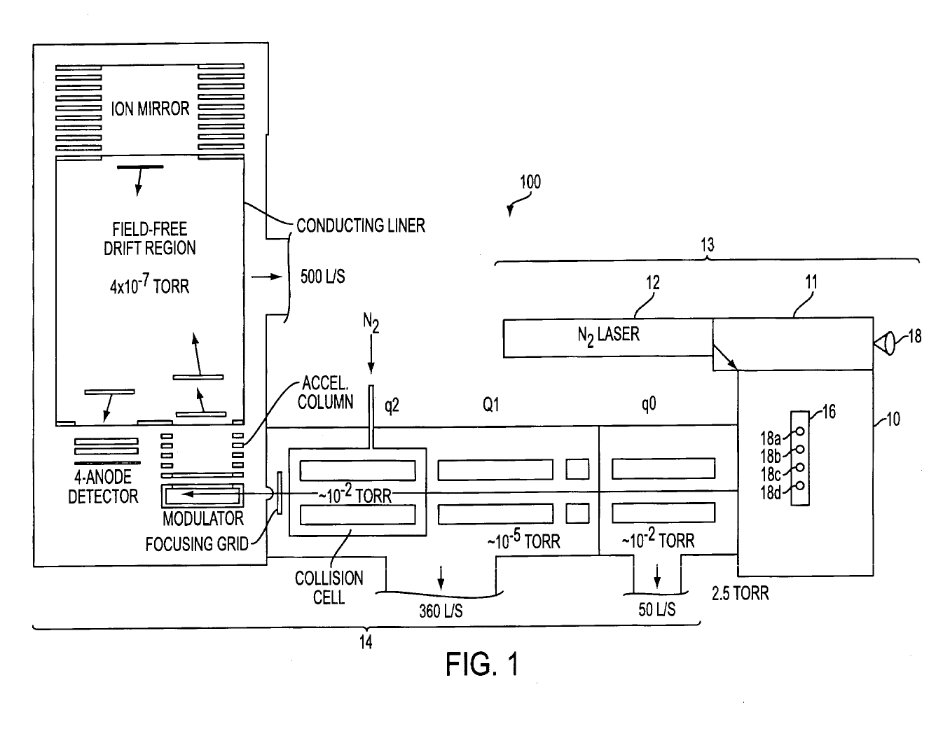

Briefly, with reference to FIG. 1, instrument 100 comprises laser desorption/ionization source 13; affinity capture probe interface 10, and tandem mass spectrometer 14. Shown in FIG. 1 is a preferred embodiment in which laser source 12 is a pulsed nitrogen laser and tandem mass spectrometer 14 is an orthogonal quadrupole time-of-flight mass spectrometer (QqTOF) tandem MS.

Laser Desorption/Ionization Source

Laser desorption/ionization source 13 produces energetic photons that, properly conditioned and directed, desorb and ionize proteins and other analytes adherent to affinity capture probe 16. Laser desorption/ionization source 13 comprises laser source 12, laser optical train 11, and, optionally, probe viewing optics 18.

Laser desorption/ionization source 13 produces pulsed laser energy either through use of a pulsed laser 12 or, alternatively, by mechanically or electronically chopping the beam from a continuous laser 12. Typically, pulsed lasers are preferred. Preferred pulsed laser sources include nitrogen lasers, Nd:YAG lasers, erbium:YAG lasers, and CO₂ lasers. Presently preferred is a pulsed nitrogen laser, due to simple footprint and relatively low cost.

Photons emitted from laser 12 are directed to strike the surface of probe 16 by laser optical train 11. Optical train 11 can consist of an arrangement of lenses, mirrors, prisms, attenuators, and/or beam splitters that function to collect, direct, focus, sub-divide, and control the intensity of each laser pulse so that an appropriate desorption fluence in the form of a focused spot of desorption energy is delivered to probe 16.

Alternatively, optical train 11 can consist of a fiber optic array that functions to collect, direct, and sub-divide the energy of each laser pulse.

In this embodiment, the output of laser 12 is coupled to the input side of an optical fiber using an optical coupler; the coupler is typically comprised of a lens whose focal length and diameter is appropriate for the input numerical aperture of the fiber.

The amount of energy entering the fiber can be controlled by prudent adjustment of the lens position with respect to the fiber; in this instance, the fiber optical coupler can double as an optical attenuator. In another preferred arrangement, the total output energy of the laser is coupled into the fiber and an attenuator is placed between the output side of the optical fiber and the desorption spot focusing elements of the optical train. In yet another preferred arrangement, an optical attenuator is placed between the laser and the optical fiber coupler. In all instances, optical attenuation is employed to insure the delivery of appropriate laser fluence to the surface of probe 16 independent of the output energy of laser 12. Typical laser fluences are on the order of 20-1000 μjoules/square millimeter.

As it is well established that fiber optic components can often be damaged when accepting focused energy from lasers, it is advantageous to maximize the acceptance area of the input side of the fiber so that the fluence of the incident laser energy is below the damage threshold of the fiber. The latter also simplifies alignment of the laser beam with the optical fiber when adjusting the relative position of the optical coupler with respect of the laser and optical fiber. However, in order to obtain reasonable desorption fluence levels at probe 16, a maximum exit side fiber diameter of 400 μm (microns) should not be exceeded when used with typical nitrogen lasers delivering a maximum energy of about 200 μJ/laser pulse. A solution to this problem lies in the incorporation of a tapered optical fiber whose input side has a diameter on the order of 400 to 1200 microns and output side has a diameter of 200 to 400 microns.

Typically, the desorption spot should be focused to a size that maximizes the generation of ions for each pulse by interrogating the greatest area of probe 16 while maintaining sufficient fluence to induce desorption and ionization. While using typical nitrogen lasers delivering a maximum energy of about 200 μJ/pulse in a laser desorption/ionization source coupled to a quadrupole-quadrupole time-of-flight tandem mass spectrometer, an optimum laser spot area has been determined to range between 0.4 and 0.2 square millimeters.

Laser desorption/ionization source 13 can include, typically as an integral part of optical train 11, probe viewing optics 18. Viewing optics 18 can contain an illumination source, lenses, mirrors, prisms, dichroic mirrors, band-pass filters, and a CCD camera to allow the illumination and viewing of the desorption locus, i.e., the region of probe 16 to be interrogated by laser.

Where laser optical train 11 comprises an optical fiber, viewing optics 18 can take advantage of light from the optical fiber itself.

For example, the fiber optic coupler can be bifurcated to split off a small fraction of the laser excitation energy to be used as a means of monitoring the applied laser energy, or it can be bifurcated to allow the introduction of visible light to illuminate the desorption locus.

In the first of these two embodiments, a small fraction of the excitation energy is directed to impinge upon a photo-detector that is an integral component of a laser energy circuit calibrated to reflect the actual amount of laser energy delivered to probe 16. In the second embodiment, visible light is directed to illuminate the desorption locus making viewing of this region possible, either through a separate set of photo optics coupled to a CCD camera or by the employment of a prism or dichroic mirror, between the optical fiber and the laser excitation source, that directs light reflected up the main branch of the optical fiber towards a CCD camera. Alternatively, a prism or dichroic mirror can be placed in line between the illuminating fiber branch of the optical fiber and the illumination source to allow any back reflected images that couple into this branch to be directed to impinge upon a CCD camera. In yet another embodiment, the fiber can be trifurcated so that one branch delivers desorption/ionization laser pulses, the second branch delivers visible light for illuminating the desorption locus, and the third branch transmits reflected light from the desorption locus to a CCD camera. For each of these viewing schemes, an appropriate band-pass filter should be deployed between the CCD camera and viewing optical train to prevent the transmission of possibly damaging high energy photons that arise as the direct reflection of the incident laser pulse upon the probe surface or are secondary photons emitted from the probe surface as a direct consequence of electronic excitation by the incident laser pulse.

Probe Interface

Affinity capture probe interface 10 is capable of reversibly engaging affinity capture probe 16, positioning probe 16 in interrogatable relationship to laser source 12 and concurrently in communication with tandem mass spectrometer 14; the communication supports atmospheric to subatmospheric pressure.

Probe interface 10 comprises a probe holder, probe introduction port, probe position actuator assembly, vacuum and pneumatic assembly, and an interface ion collection system.

The probe holder is a component of probe interface 10 shaped to conform to the form factor of probe 16. Where probe 16 is a ProteinChip® Array (Ciphergen Biosystems, Inc., Fremont, Calif. USA), the probe holder conforms to the form factor of the ProteinChip® Array.

The probe holder can hold a single probe 16 or a plurality of probes 16. The holder positions each probe 16 in proper orientation to be interrogated by laser desorption/ionization source 13 and with respect to the interface ion collection system.

The probe holder makes intimate contact with a position actuator assembly.

The actuator assembly moves the relative position of probe 16 with respect to laser desorption/ionization source 13 and the interface ion collection system so that different regions of the probe can be interrogated and ions resulting from such irradiation collected for introduction into tandem mass spectrometer 14.

The actuator consists of electro-mechanical devices that support translational and/or rotational movement of probe 16 while maintaining the probe's position with respect to the laser desorption/ionization source and ion collection system constant. Such electromechanical devices include but are not limited to mechanical or optical position sensors, solenoids, stepper motors, DC or AC synchronous motors that either directly or indirectly communicate with linear motion actuators, linear or circular motion guide rails, gimbals, bearings, or axles.

A probe introduction port allows the probe holder, containing loaded probes 16, to be placed onto the probe position actuator assembly without introducing undue levels of atmospheric gas into the probe interface 10 and tandem mass spectrometer 14.

In order to accomplish the latter, the probe introduction port uses a vacuum evacuation system (the probe introduction port evacuation system) to pump out atmospheric gas, achieving a target port pressure prior to moving the chip into the working position. During probe exchange, the probe actuator assembly moves the probes from the working position (that position in alignment with laser desorption source 13 and the ion collection system) to an exchange position. In doing so, the actuator can provide a seal between the exchange port that is soon to be raised to atmospheric pressure, and the inlet of the mass spectrometer. After sealing off the mass spectrometer inlet, atmospheric gas is introduced into the probe introduction port by a probe introduction port pressurization system. This eliminates the pressure difference between the atmospheric surface of the probe holder and the introduction port, allowing the probe holder to be removed from the probe position actuator assembly.

Following the removal of previously analyzed probes 16 and the installation of new probes 16, the probe holder is replaced into its position actuator and the sample loading process begins. As previously described, the probe introduction port can be pumped down to sub-atmospheric pressure by the evacuation system. Upon achieving the target sample introduction pressure, the probe actuator system moves probe 16 from the exchange position to the working position, and in doing so opens the seal to the mass spectrometer inlet.

Where, alternatively, ions are generated in a desorption chamber held at atmospheric pressure and ultimately directed to an ion optic assembly that introduces the ions to the mass spectrometer inlet, it is not necessary to evacuate and pressurize the probe introduction port since it will be maintained at atmospheric pressure.

The probe introduction port evacuation system consists of a vacuum pump, pressure sensor, vacuum compatible tubing and connecting fittings, as well as vacuum compatible valves that, when acting in concert, allow the controlled evacuation of atmospheric gas contained within the introduction port following sample exchange so that probes 16 can be moved into the working position. The vacuum pump can be, but is not limited to, a single stage or multi-stage oil mechanical pump, a scroll pump, or oil-free diaphragm pump. In a preferred embodiment, the vacuum compatible valves are electrically controlled solenoid valves. In the same embodiment, the pressure sensor is an electronic sensor capable of operating in pressure domains ranging from atmospheric pressure to 1 millitorr. Such pressure sensors include but are not limited to thermocouple gauges and pirani gauges. In the same embodiment, concerted operation of this system is achieved under logic controlled provided by an analog logic circuit or digital microprocessor that reconciles inputs from the pressure gauge and positional sensors to allow for automated evacuation of the sample port as part of the overall instrument operation.

The probe introduction port pressurization system consists of a gas source, pressure sensor, gas conducting tubing and fittings, and gas compatible valves that, when acting in concert, allow the controlled introduction of gas that pressurizes the exchange port, thus allowing removal of the probe holder from the actuator assembly.

In one embodiment, the gas source is untreated atmospheric gas. In another embodiment, the gas source is atmospheric gas that is first directed through a moisture absorbent trap and optionally secondly through a particulate filter prior to introduction to the pressurization system. In another embodiment, pressurizing gas is supplied by a purified source of dry inert gas such as nitrogen or any of the cost-effective noble gases in lieu of using atmospheric gas.

In a preferred embodiment, the gas conducting tubing, fittings, some of the valves, and pressure sensor of the pressurization system are those used in the evacuation system. In the same embodiment, concerted operation of this system is achieved under logic control provided by an analog logic circuit or digital microprocessor that utilizes inputs from the pressure gauge and positional sensors to allow for automated pressurizing of the sample port as part of the overall instrument operation.

The probe interface pressure regulation system functions to provide selective background gas pressure in the desorption chamber that exists between the sample presenting (adsorption) surface of probe 16 and the ion collection system. Acceptable desorption chamber pressure ranges extend from atmospheric pressure to 0.1 microtorr. A preferred pressure range extends from 1 torr to 1 millitorr. The probe interface pressure regulation system consists of a gas source, gas conducting tubing and fittings, a gas flow regulator, and a pressure sensor. The gas source can be untreated atmospheric gas. In another embodiment, the gas source is atmospheric gas that is first directed through a moisture absorbent trap and optionally secondly through a particulate filter prior to introduction to the regulation system. In another embodiment, regulation gas is supplied by a purified source of dry inert gas such as nitrogen or any of the cost-effective noble gases. The gas flow regulator may be a manually controlled flow restrictor. Alternatively, gas flow regulation may be achieved by using an electronically controlled flow restrictor. In a preferred embodiment, close loop control of preferred desorption chamber pressure is achieved in an automated fashion under logic control provided by an analog logic circuit or digital microprocessor that actively interacts with an automated gas flow regulator to achieve a pre-established reading from the pressure gauge.

The interface ion collection system consists of electrostatic ion collection assembly, an optional pneumatic ion collection assembly, and an electrostatic or RF ion guide. The electrostatic ion collection assembly consists of an arrangement of DC electrostatic lens elements that function to collect ions desorbed within the desorption chamber and direct them towards the mass spectrometer inlet.

In one embodiment, this assembly consists of two electrostatic elements. The first element is comprised of the probe holder and probe surface and the second is an extractor lens. The extractor lens is arranged to be between 0.2 to 4 mm away from the surface of the array. The extractor lens contains an aperture ranging from 2 mm to 20 mm in diameter that is concentrically located about a normal axis that extends from the center of the desorption locus to the center of the mass spectrometer inlet. Independent DC potentials are applied to each element of this assembly.

In a preferred embodiment, the extractor lens contains a 10 mm diameter aperture and is located 1 mm away from the array surface. In the same preferred embodiment, a ten volt potential difference is established between the extractor and array.

The pneumatic ion collection assembly consists of a gas source, conducting tubing, tubing connectors, gas flow regulators, gas pressure sensors, and a gas emission port so that a predetermined flow of gas can be created to assist the bulk transfer of desorbed ions within the desorption chamber into the mass spectrometer inlet.

The gas source can be untreated atmospheric gas. In another embodiment, the gas source is atmospheric gas that is first directed through a moisture absorbent trap and optionally secondly through a particulate filter prior to introduction to the system. In another embodiment, ion collection gas is supplied by a purified source of dry inert gas such as nitrogen or any of the cost-effective noble gases.

The gas flow regulator can be a manually controlled flow restrictor. Alternatively, gas flow regulation can be achieved by using an electronically controlled flow restrictor. The pressure sensor can be but is not limited to thermocouple gauges and pirani gauges. The gas emission port is located behind probe 16 to induce bulk gas flow around the probes and down the normal axis centrally located between the desorption locus and the mass spectrometer inlet.

In a preferred embodiment the flow of gas is under automatic closed loop control by the use of analog or digital control circuitry so that an adequate ion-sweeping flow is generated without over-pressurizing the desorption chamber.

The final component of the interface ion collection system is the ion guide. The ion guide functions to transfer the collected ions into mass spectrometer 14. It can be of the electrostatic or RF variety. A preferred embodiment is a multipolar RF ion guide. An example of the latter is a quadrupole or hexapole ion guide. In the preferred Qq-TOF instrument described in greater detail below, the ion guide is a quadrupole RF ion guide. Ions are directed into the ion guide by electrostatic and pneumatic accelerative forces, respectively created by the electrostatic and pneumatic ion collection systems. In a preferred embodiment the DC electrostatic potential of the ion guide is less than that of the extractor lens by typically 10 to 20 volts.

Tandem Mass Spectrometer

The analytical instrument of the present invention further includes tandem mass spectrometer 14. Tandem mass spectrometer 14 can usefully be selected from the group that includes orthogonal quadrupole time-of-flight (Qq-TOF), ion trap (IT), ion trap time-of-flight (IT-TOF), time-of-flight time-of-flight (TOF-TOF), and ion cyclotron resonance (ICR) varieties.

Presently preferred, and further described in detail below, is an orthogonal Qq-TOF MS.

The major strengths of the QqTOF MS are outstanding mass accuracy and resolving power; enhanced sensitivity in the peptide and low mw range; and superior ms/ms performance by employing low energy collision induced dissociation (CID). An orthogonal QqTOF with electrospray ionization source is available commercially from AB/MDS Sciex (QSTAR™; AB/MDS-Sciex, Foster City, Calif., USA).

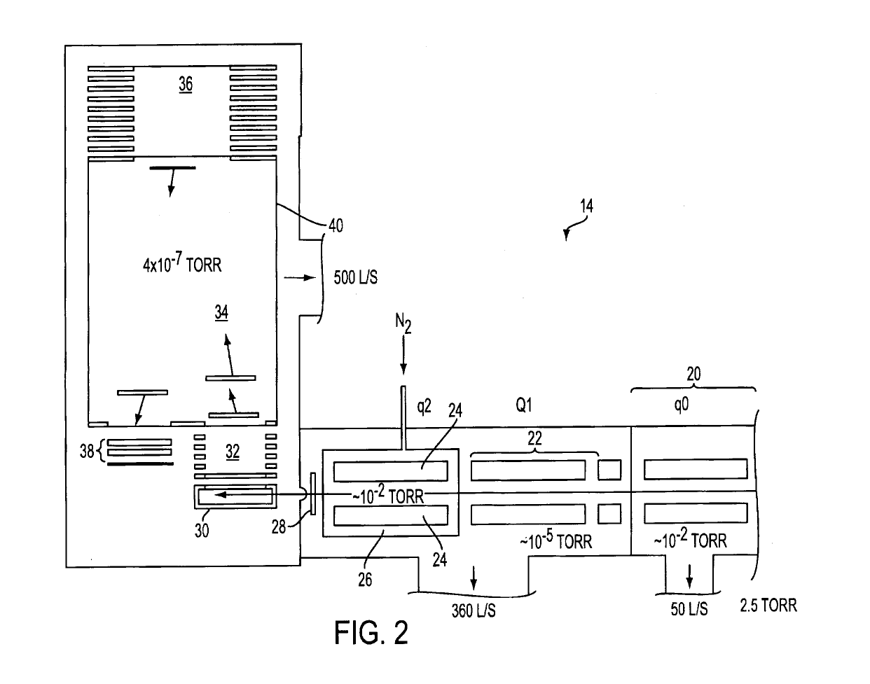

With reference to FIG. 2, the principles and features of the QqTOF will be briefly outlined.

Ions are created in a desorption chamber prior to the first quadrupole lens “q0”. Pressure within q0 is typically maintained at about 0.01 to 1 torr, but can also be maintained at atmospheric pressure. In this manner, desorbed ions are rapidly cooled by collisions with the background gas shortly after their formation.

This cooling or damping of the ion population provides three major advantages.

First, the cooling eliminates the initial energy distributions of the desorbed ions and reduces their total energy down to a point that approximates their thermal energy. This simplifies the orthogonal extraction requirement, compensating for variations in ion position and energy, thus improving ultimate resolving power. A direct consequence of this improved resolution is enhanced mass accuracy down to the low ppm level.

The second major advantage of collisional cooling is its ability to decrease the rate of long term ion decay. Gas collisions relax internal excitation and improve the stability of peptide and protein ions. This stabilizing effect appears to be maximized when ions are created in the presence of about 1 torr pressure of background gas. Measurements published by others have indicated that losses of small groups and background fragmentation can be practically eliminated, improving the transmission of high mw proteins and other labile biopolymers (i.e. glyco-conjugates, DNA, etc.). Faster decay mechanisms (prompt and in-source type decay) still occur.

The final advantage of q0 collisional cooling is in the creation of a pseudo-continuous flow of ions into the mass analyzer. Ion collisions in q0 cause the desorption cloud to spread out along the axis of q0. This spreading creates a situation in which ions from various desorption events begin to overlap, creating an electrospray-like continuous introduction of ions into the analyzer.

After passing through q0, ions enter a second quadrupole 22 (“Q1”). This quadrupole functions as either an ion guide or as a mass filter. It is here that ion selection is created for ms/ms or single ion monitoring (SIM) experiments.

After exiting Q1, ions enter a third quadrupole 24 (“q2”) positioned in collision cell 26. During simple experiments, q2 is operated as a simple rf ion guide. For ms/ms experiments, q2 is filled with collision gas at a pressure of about 10⁻² torr to promote low energy CID.

After exiting q2, ions are slightly accelerated by a DC potential difference applied between the exit of q2 and focusing grid 28. This acceleration “biases” the velocities of the ions in the Y-axis so that their velocities are now inversely related to the square root of their m/z. This must be accomplished if all ions of different m/z are to strike the detector after orthogonal extraction and free flight. If such biasing is not accomplished, ions of different m/z will enter the orthogonal extraction region with the same Y-axis velocity.

As always in time-of-flight, ions of lower m/z will strike the detector before ions of greater m/z. The absolute degree of displacement in the Y-axis will be a product of an ion's flight time in the Z-axis and an ion's Y-axis velocity. If the detector is placed at some location optimized for intermediate mw ions, lighter ions will “undershoot” the detector arriving to the right side of the detector in FIG. 2. Conversely, ions of greater m/z will “overshoot” the detector and arrive at the left side of the detector in FIG. 2. Consequently, it is necessary for all ions to maintain a constant ratio of Z- and Y-axis velocities if all ions are to strike a common detection point. The previously described grid biasing method accomplishes this.

After passing through focusing grid 28, ions arrive in modulator region 30 of the orthogonal extraction elements. Modulator 30 is pulsed at rates approaching 10,000 pulses/second (10 kHz). Ions are pushed into accelerator column 32 of the ion optic and exit out into free flight region 34 of the orthogonal time-of-flight (O-TOF). Energy correction is achieved when the ions enter ion mirror 36. In the mirror, ions are turned around and are directed to strike fast response, chevron array microchannel plate detector 38.

Alternatives to this prototypical arrangement can be used.

For example, the geometry presented above presents the difficulty of performing O-TOF at high acceleration energies. It is well established that ion detection sensitivity for peptides and proteins is improved as total ion energy increases. For human insulin (MW=5807.65 Da), detection efficiency approaches 100% at ion energies of 35 keV when using typical microchannel plate detectors. If the ions are to be accelerated to 20 or 30 keV of energy, free flight tube liner 40 and other corresponding components must be floated to −20 kV or −30 kV, respectively. The difficulties in providing stable electrical isolation on simple ion optic elements at such potentials are well known. To safely and reliably float a plurality of elements at such potentials is difficult. One solution is the employment of post-acceleration technology.

Unlike the device described above, such an alternative device employs a detector post accelerator (not shown). Ions are accelerated to about 4 keV of energy after leaving the orthogonal extraction elements and the free flight region is floated at −4 kV. Further acceleration is achieved as ions enter a post-accelerator detector assembly. In this assembly, ions pass through a field-retaining grid held at liner potential. Ions then receive additional acceleration in a field established between the field-retaining grid and the primary ion conversion surface of the detector. Such acceleration fields are on the order of 10 to 20 kV over 4 to 10 mm distances.

Because the orthogonal design uncouples the time of flight measurement from ion formation, a number of advantages are realized.

Laser fluence related problems, such as peak broadening due to ion shielding and ion acceleration field collapse, are eliminated because ions of the desorption plume have an extended period of time (typically a few milliseconds) to expand and cool prior to orthogonal extraction and acceleration into the TOF mass analyzer. Additionally, orthogonal extraction eliminates much of the large hump and baseline anomaly seen at the beginning of high laser energy, conventional extraction spectra due to the chemical noise created by the excessive neutral load of the EAM. Because neutrals are not extracted in the modulator region, only ions are transmitted down to the detector and chemical noise is appreciably reduced.

These factors allow the use of laser fluences that are 2-3 times greater than those normally employed during parallel continuous or delayed ion extraction approaches. The net result is an almost complete elimination of the need to hunt and search for “sweet spots” even in the presence of poor sample-EAM homogeneity, as well as improved external standard mass accuracy determination (typical errors are between 20-50 ppm), improved quantitative reproducibility, and improved signal to noise. An additional benefit is the elimination of the need to perform low and high laser energy scans to analyze ions of a broad m/z range. A single laser fluence can now be employed to see both low and high mw ions, greatly simplifying the analysis of unknown mixtures.

Perhaps one of the most impressive advantages of this device when compared to conventional parallel extraction approaches lies in its ability to obviate the need for rigid sample positioning requirements. Because the TOF measurement is substantially removed from the ion formation process, the original position of the ion is no longer important. Furthermore, since ion formation is accomplished in a high-pressure environment without concomitant application of high voltage extraction fields, the design requirements of solid-state sample inlet systems are greatly relieved. Simple approaches can be taken to employ 2-dimensional sample manipulators while maintaining excellent, external-standard mass accuracy performance. Additionally, sample presenting surfaces no longer need to be made of metals or other conductive media.

To summarize, the laser desorption ionization (LDI) Qq-TOF MS has the following advantages over existing LDI-TOF MS technology: (1) increased external standard mass accuracy (20-50 ppm typical); (2) enhanced resolution; (3) improved ms/ms efficiency; (4) improved ease of signal production using a single high laser energy level that eliminates the need for high and low energy scans; (5) improved quantitative ability through the use of TDC technology and laser fluences 2-4 times above minimum desorption threshold; (6) reduced requirements for 2-dimension sample actuators; (7) potential for using plastic components for sample presenting probe surfaces (injection molded two dimensional probe arrays, for example); (8) reduced chemical noise by using single ion monitoring and enhanced ability to measure for ions in the EAM chemical noise domain.

The laser desorption ionization (LDI) Qq-TOF MS has the following advantages over existing MALDI-PSD approaches in protein characterization and identification.

The LDI-QqTOF provides higher mass resolving power and mass accuracy; in database mining approaches, this increased capability reduces the number of false positive database hits, simplifying identification. Furthermore, the QqTOF also provides greater than an order of magnitude greater sensitivity than can be obtained with PSD MS/MS.

The analytical instrument of the present invention demonstrates impressive MS/MS capability and less than 20 ppm mass assignment error for single MS analysis. The latter has allowed the identification of a number of proteins simultaneously retained on the surface of a single affinity capture probe.

Other Components

Affinity capture probe tandem MS instrument 100 typically further comprises a digital computer interfaced with the tandem mass spectrometer detector. The digital computer is typically further interfaced with laser desorption source 12, permitting the computer both to control ion generation and to participate in data acquisition and analysis.

Analysis software can be local to the computer or can be remote, but communicably accessible to the computer. For example, the computer can have a connection to the internet permitting use of analytical packages such as Protein Prospector, PROWL, or the Mascot Search Engine, which are available on the world wide web. The analysis software can also be remotely resident on a LAN or WAN server.

Affinity Capture Probes

To conduct analyses, such as those described in detail in sections herein below, at least one affinity capture probe 16 having adsorbed analyte is engaged in probe interface 10 in position to be interrogated by laser desorption/ionization source 13 and to deliver desorbed ions into tandem mass spectrometer 14.

Probes 16 typically have one or more adsorption surfaces 18, which surfaces can differ from one another (18a, 18b, 18c, 18d). Typically, if there are a plurality of adsorption surfaces 18, all are exposed on a common face of probe 16.

Adsorption surfaces 18 are typically either chromatographic adsorption surfaces or biomolecule affinity surfaces.

Chromatographic affinity surfaces have an adsorbent capable of chromatographic discrimination among or separation of analytes. Such surfaces can thus include anion exchange moieties, cation exchange moieties, reverse phase moieties, metal affinity capture moieties, and mixed-mode adsorbents, as such terms are understood in the chromatographic arts. Biomolecule affinity surfaces have an adsorbent comprising biomolecules capable of specific binding. Such surfaces can thus include antibodies, receptors, nucleic acids, lectins, enzymes, biotin, avidin, streptavidin, Staph protein A and Staph protein G. Adsorbent surfaces are further described in a section below.

Interface 10 positions probe 16 in interrogatable relationship to laser desorption/ionization source 13. Typically, it is desired that the laser interrogate probe adsorption surfaces 18. Accordingly, interface 10 positions probe 16 adsorption surfaces 18 in interrogatable relationship to laser desorption/ionization source 13. If adsorption surfaces 18 are positioned on only one face of probe 16, probe 16 and/or the probe holder of interface 10 can be asymmetrically dimensioned, thus obligating insertion of probe 16 in the orientation that presents adsorption surfaces 18 to laser desorption source 13.

Where probe 16 has a plurality of adsorption surfaces 18, it will be desired that laser source 12 be able discretely to address each adsorption surface 18. This can be accomplished by optics interposed between laser source 12 and interface 10, by rendering laser source 12 and/or interface 10 movable, or by a combination thereof.

Probe 16 can be an affinity capture probe as presently used in single MS analysis (e.g., those commercially available from Ciphergen Biosystems, Inc., Fremont, Calif. USA).

III. Applications of the Affinity Probe Tandem MS Instrument

The above-described analytical instrument of the present invention provides significant advantages in, and affords novel methods for, (1) protein discovery and identification and (2) the characterization of interactions between specific binding pairs, which will now be described in turn.

In general, the advantages of the above-described analytical instrument include: the ability to do high mass accuracy measurements in single mass MS and tandem MS mode combined with affinity capture probe technology, especially with a specific receptor binding system.

A. Protein Discovery and Identification

1. Advantages of the Methods of the Invention

One related set of problems that protein biologists attempt to solve is protein discovery, identification, and assay development. Protein discovery is the process of finding proteins in a system that are biologically interesting because, for example, they function as diagnostic markers or carry out critical cell functions. Protein identification is the process of determining the identify of a discovered protein. Assay development is the process of developing a reliable assay to detect the protein. The methods of this invention provides advantages for the practitioner in carrying out these processes compared with previous technologies.

A primary advantage of this invention is that it provides a single platform on which to carry out process steps from protein discovery to protein identification to assay development. The provision of a single platform based on SELDI technology significantly decreases the time between discovery and assay validation: What used to takes months using previous technologies can now take weeks or days. The methods of this invention also significantly reduce the amount of sample required to perform the experiments. Whereas previous methods required micromoles of analyte, the present methods can perform the same experiments with picomoles of analyte. This overcomes a significant hurdle when sample is scarce or scale-up is difficult.

Previously, protein discovery and isolation was accomplished using 2D gels or Western Blots. However, comparison of gels to each other to detect differentially expressed proteins is a difficult procedure.

The discovered protein might now be identified using mass spectrometry methods. Important proteins could be isolated and ultimately fragmented in the gel with proteases and the peptide fragments could be analyzed by a mass spectrometer and appropriate bioinformatics methods. However, gels are not compatible with present mass spectrometry methods, and peptide fragments have to be removed from the gel. Because the latter process inevitably resulted sample loss, this approach required large quantities of starting protein and material. When the protein was rare, as important proteins can be, this increased the difficulty of the process.

Once identified, the practitioner needs to develop a reliable assay to detect the protein. Typically, this involves developing an ELISA assay. This technology, in turn, required the production of antibodies. This can be a time consuming task, especially if the protein of interest if difficult to produce in quantity for immunization.

Thus, prior techniques could have required three different technologies to accomplish protein discovery, protein identification and protein assay. The methods of the present invention can accomplish this with one technology.

2. Methods of Protein Discovery, Identification and Assay Development

The methods of this invention for protein discovery, identification and assay development involve preparing a difference map to discover a protein or proteins of interest, identifying the protein by affinity capture probe tandem MS, and validating using an affinity capture probe laser desorption ionization chromatographic surface assay or affinity capture probe laser desorption ionization biospecific surface assay.

The process can proceed as follows. A protein of interest is provided or is discovered by, for example, using difference mapping of retentate studies. These methods are described in, for example, WO 98/59362 (Hutchens and Yip). Briefly, two biological samples that differ in some important respect (e.g., normal v. diseased; functional v. non-functional) are examined by retentate chromatography methods. The methods involve exposing the samples to a plurality of different chromatographic affinity and wash conditions, followed by examination of the “retained proteins” by affinity capture probe laser desorption ionization. Proteins that are differentially expressed between the two samples are candidates for further examination. Because they have been examined on a mass spectrometer, the molecular weights of these candidate proteins are known.

Normally, scores of proteins in addition to the proteins of interest will be retained on the chip. Therefore a next optional step is to refine the affinity and wash conditions under which the protein or proteins of interest are retained so as to simplify the sample for further analysis. (These also are described in the Hutchens and Yip application.) While capture of the single protein of interest is ideal, capture of no more than about ten detectable proteins is favorable. The refined method provides an improved chromatographic assay for the protein of interest.

The retained proteins are then subject to fragmentation on the probe using a proteolytic agent of choice, producing a pool of peptides for subsequent study. Digestion by specific endoproteases such as trypsin is advantageous because the cleavage pattern is known and is compatible with bioinformatics methods involving in silico cleavage of proteins stored in a database. The resulting peptides are then analyzed by high resolution, high accuracy MS-MS (e.g., having a mass assignment error of less than 20 parts per million and resolving power of approximately 10,000). At this point, it may not be clear whether a particular peptide fragment is a cleavage product of the protein of interest or of one of the other retained proteins. Nevertheless, the analysis proceeds by selecting one of the peptide fragments (possibly at random, possibly based on information that it corresponds to the protein of interest) and subjecting the peptide to gas phase fragmentation. One such method is collision-induced dissociation (CID). The peptide need not be isolated from the chip, because the MS-MS device isolates the peptide of interest from the other peptides in the mass spectrometer. This will generate a further fragmentation pattern of the selected peptide fragment.

Using methods already established in the art, such as database mining protocols, information from the fragmentation pattern is used to interrogate a protein database to generate one or more putative identify candidates for the protein from which the peptide fragment is derived. The protocols generally perform a closeness-of-fit analysis that measures how well the predicted mass spectrum of a protein matches the actual mass spectrum of the selected fragment. Proteins in the database can then be ranked based on the confidence measurement that the protein fragment corresponds to a database protein. Knowledge of the mass of the parent protein and the species of origin, both of which are already known, will assist in limiting the number of identify candidates generated.

Then, the putative identity of the protein from which the peptide fragment was generated is verified. Using knowledge from the database of the primary sequence of the putative identity candidate and the cleavage pattern of the proteolytic agent used, one can predict the peptide fragments and, in particular, their molecular weights, that should be generated from the cleavage of the identity candidate by the proteolytic agent. This predicted set of fragments is then compared with the actual set of fragments generated after proteolytic cleavage of the proteins retained on the chip based on their masses. If the predicted fragments are accounted for, then one is confident that the putative identity candidate actually corresponds to the identify of one of the proteins retained on the chip. If not, then one must test other putative identity candidates through a process of elimination until the protein from which the fragment is generated is identified. At this point, the generated fragments that correspond to the identified protein can be eliminated from the total set of fragments generated as having been accounted for.

If only one protein was retained after refining the affinity and wash conditions, then all the peptide fragments will have been accounted for and the process is complete. However, if more than one protein has been retained, the situation may be more complicated. For example, the fragment used in the analysis may have been generated from the protein of interest, or it may have been generated by a protein that was retained on the chip, but that is not the protein of interest. In this case, it is useful to repeat the steps of analyzing the peptide fragments not accounted for by the MS-MS methods described until the protein of interest is identified or all the retained proteins have been identified.

Finally, the protein of interest can be assayed by affinity capture probe laser desorption ionization methods using either chromatographic surface already determined to retain the protein or a biospecific surface can be developed for use in an affinity capture probe laser desorption ionization assay. Creation of biospecific surfaces involves providing a binding partner for the identified protein, such as an antibody, or a receptor if a receptor is known, and attaching this to the chip surface. Then, the protein of interest can be assayed by SELDI as already described.

B. Characterization of Molecular Interactions

The analytical instrument of the present invention makes possible, for the first time, a sensitive, efficient, single-platform approach to the study of interactions between specific binding partners.

Specific binding partner interactions are at the core of a wide spectrum of biological processes. Accordingly, the ability to measure and to characterize such interactions is a necessary prerequisite to a full understanding such processes; at the clinical level, the ability to measure and to characterize such interactions is important to an understanding of pathologic aberrations in those processes, and to the rational design of agents that can be used to modulate, or even abrogate, such interactions.

At the level of organized eukaryotic tissues, for example, intercellular signaling in the mammalian nervous system is mediated through interactions of neurotransmitters with their cognate receptors. An understanding of the molecular nature of such binding interactions is necessary for a full understanding of such signaling mechanisms. At the clinical level, an understanding of the molecular nature of such binding interactions is required for a full understanding of the mechanism of signaling pathologies, and for the rational design of agents that palliate such signaling pathologies, agents useful for treatment of diseases ranging from Parkinson's disease to schizophrenia, from obsessive compulsive disorder to epilepsy.

At the circulatory level, for example, interaction of B cell receptors with circulating antigen is required to trigger B cell clonal expansion, differentiation, and antigen-specific humoral immune response. An understanding of the antigenic epitopes that contribute to antigen recognition is critical to a full understanding of immune responsiveness. At the clinical level, such understanding is important to the design of vaccines that confer more robust humoral immunity. Analogously, interaction of T cell receptors with peptide displayed in association with MHC on antigen-presenting cells is critical to the triggering of cellular immunity. An understanding of the T cell epitopes that contribute to antigen recognition is important to the design of vaccines that confer more robust cellular immunity.

At the level of individual cells, phenotypic response to extracellular signals is mediated by at least one, most often a cascade of intermolecular interactions, from the initial interaction of a cell surface receptor with ligand, to intracytoplasmic interactions that transduce the signal to the nucleus, to interaction of protein transcription factors with DNA, the altered patterns of gene expression then leading to the observed phenotypic response.

For example, discriminative binding of estrogen and progesterone by ovarian cells is required for ovulation. An understanding of the molecular nature of binding interactions between steroid hormone receptors and the hormone ligand, on the one hand, and liganded receptor with steroid hormone response elements in the genome, on the other, is important for an understanding of the hormonal response. Such understanding, in turn, is important for an understanding of infertility, and for the rational design of agents—such as RU486—that are intended to abrogate ovulation, implantation, and/or fetal viability.

Such interactions are found not only in eukaryotic systems, but in prokaryotic systems and in the interaction of prokaryotes with eukaryotes. For example, certain gram negative bacteria elaborate a pilus that is required for invasion of the eukaryotic eurethra; and understanding of such interaction is important to full comprehension of the pathologic process, and for the rational design of agents that can prevent such invasion.

A number of techniques are used in the art to study and map such intermolecular interactions between specific binding partners. Each has significant disadvantages.

In a first such method, one member of a specific binding pair is immobilized on an adsorbent which is packed in chromatographic column. To map the location within the structure of the second (free) binding partner that makes contact with the first (bound) binding partner, the second (free) partner is cleaved. Typically, such cleavage is by specific proteolytic enzyme, although specific chemical cleavage (e.g., by CNBr) or even nonspecific chemical hydrolysis can be done. Thereafter, the digest is passed over the column to bind those portions of the second (free) partner that still bind to the first (immobilized) partner.

The peptides of the second partner are then eluted, typically using a salt or pH gradient, and identified, typically by introducing the peptides into a mass spectrometer using by MALDI or electrospray ionization.

This approach has several well known, and significant, problems. First, a large quantity of purified first binding partner is required in order to create the specific adsorbent. Second, a large quantity of second binding partner, typically purified, is required for digestion, adsorption, and elution, since each of these stages is attended by dilution effects and analyte loss. Furthermore, although the subsequent mass spectrometric analysis can be highly sensitive, interfacing the fluid phase analysis to MS can also occasion analyte loss.

Perhaps a more fundamental disadvantage is that, by cleaving the second binding partner before binding to the first partner, only those molecular structures on the second binding partner that are properly maintained in the peptide fragments will bind, and thereafter be detected. If, for example, an antibody binds antigen at discontinuous, rather than linear, epitopes, such discontinuous epitopes can be destroyed by fragmentation; unable to support binding to the immobilized antibody, such antigenic epitopes cannot be detected.

A second typical approach in the art is to use point mutations to map, within a protein binding partner, those residues that contribute to intermolecular binding.

This latter approach requires that the protein binding partner have been cloned, the creation of the desired point mutations, recombinant expression of the altered protein, and purification thereof. Thereafter, the binding kinetics to the other partner of the altered protein is measured to determine the effect of the mutated residue on the intermolecular interaction.

Less often used, the nature of the contacts between binding partners can be elucidated by X-ray crystallography of the bound partners. This technique is highly effective, and provides atomic level resolution, but requires that each binding partner be highly purified, and further requires that suitable co-crystals be formed.

The affinity capture tandem mass spectrometry instrument of the present invention provides an improved approach that requires far less starting material, obviates point mutational analysis, crystallization, and substantially reduces the purity requirement.

The first step is to immobilize one of the binding partners on an affinity capture probe.

Either partner can be immobilized; it is the free partner, however, for which structural information about the binding contacts will be obtained. Using receptor/ligand interactions as exemplary of the approach, immobilizing the ligand on the probe will permit the identification of regions of the receptor that participate in binding the ligand; immobilizing the receptor on the probe will permit the identification of regions of the ligand that participate in its binding to the receptor. Where the ligand is a protein—for example a protein hormone, cytokine, or chemokine—separate experiments, using each partner in turn, will yield a bilateral understanding of the intermolecular contacts.

The probe-bound partner can be immobilized using covalent or strong noncovalent interactions. The choice will depend upon the availability of suitable reactive groups on the partner to be immobilized and on the chemical nature of the surface of the probe. Appropriate chemistries are well known in the analytical arts.

For example, where the binding partner to be immobilized has free amino groups, covalent bonds can be formed between the free amino groups of the binding partner and a carbonyldiimidazole moiety of the probe surface. Analogously, free amino or thiol groups of the binding partner can be used covalently to bind the partner to a probe surface having epoxy groups. Strong coordinate or dative bonds can be formed between free sulfhydryl groups of the binding partner and gold or platinum on the probe surface.

Optionally, remaining reactive sites on the probe surface can then be blocked to reduce nonspecific binding to the activated probe surface.

The second (free) binding partner is then contacted to the affinity capture chip and allowed to bind to the first (immobilized) binding partner.

The second (free) binding partner can be present pure in solution, if known and available, or, more typically, will be captured from a heterogeneous mixture, such as a biological sample suspected to contain the second binding partner. The biological sample, as in biomarker discovery approaches described earlier, can be a biological fluid, such as blood, sera, plasma, lymph, interstitial fluid, urine, exudates, can be a cell lysate, a cellular secretion, or a partially fractionated and purified portion thereof.

The probe is then washed with one or more eluants having defined elution characteristics. These washes serve to reduce the number of species that bind nonspecifically to the probe.

Energy absorbing molecules are then applied, typically in the liquid phase, and allowed to dry. Application of energy absorbing molecules is effected in the same manner as for existing uses of affinity capture probes; where ProteinChip® Arrays (Ciphergen Biosystems, Inc., Fremont, Calif., USA) are used, energy absorbing molecules are applied according to manufacturer instructions.

Species that are noncovalently bound to the affinity capture probe—e.g., second binding partners specifically bound to the first (immobilized) binding partners, molecules nonspecifically bound to the probe surface, molecules nonspecifically bound to the first binding partners—are then detected in a first phase of laser desorption ionization mass spectrometry.

The mass spectrometer can be a single stage affinity capture LDI-MS device, such as the PBS II from Ciphergen Biosystems, Inc. (Fremont, Calif. USA). However, the affinity capture tandem MS of the present invention provides higher mass accuracy and higher mass resolution and is preferred.

Typically, the second (free) binding partner will be known from earlier studies, and its presence or absence readily confirmable by mass spectrometry. If the second (free) binding partner is unknown, each of the species bound to the probe can be investigated in turn. If the number of detectable species is too high, the affinity capture probe can be washed with eluants having different elution characteristics (typically, increased stringency), to reduce the number of species present for analysis.

Once binding of the second (“free”) binding partner to the first (immobilized) binding partner is confirmed, the second binding partner is fragmented. This is typically accomplished by contacting the second binding partner (which is, at this point, noncovalently but specifically bound to the first binding partner, which is, in turn, immobilized on the probe surface) with specific endoproteases, such as trypsin, Glu-C (V8) protease, endoproteinase Arg-C (either the serine protease or cysteine protease Arg-C enzyme), Asn-N protease, or Lys-C protease.

After digestion, peptides are detected by mass spectrometry.

If all fragments of the second binding partner are to be identified—e.g., to confirm the identity of the second binding partner by peptide mass fingerprint analysis—energy absorbing molecules can be applied and the probe used to introduce the peptides into a mass spectrometry by laser desorption ionization. For this purpose, the Ciphergen PBS II single acceleration stage linear TOF MS can be used; the tandem MS of the present invention, which provides superior mass accuracy and mass resolution is preferred, since the increased resolution and accuracy reduces the number of putative “hits” returned at any given confidence level in any given database query.

More typically, however, it is desired to analyze those fragments of the second binding partner that bind most tightly to the immobilized first binding partner. In such case, the probe is washed with one or more eluants prior to addition of energy absorbing molecules.

At this point, the probe is inserted into the interface of the tandem MS of the present invention, and fragments (typically peptides) of the second binding partner detected.

If the identify of the second (free) binding partner is known, the masses of the detected fragments can be compared with those predicted by applying the known cleavage rules of the fragmenting enzyme to the primary amino acid sequence of the second binding partner. In this fashion, each fragment can be identified, thus locating within the structure of the second binding partner those portions responsible for binding to the first binding partner.

Although, in theory, a single stage MS device can be used, in practice fragments other than those arising from the second binding partner will be present, confounding such analysis. Definitive identification in the usual case thus benefits from the high mass resolution and mass accuracy of the instrument of the present invention, and further often benefits from ms/ms analysis.

If the second (free) binding partner is not known, the partner can be identified by ms/ms analysis.

Typically, such analysis takes the form of selecting a first parent peptide in a first stage of MS, fragmenting the selected peptide, and then generating a fragment mass spectrum in a second stage of MS analysis. Fragmentation is done in the gas phase, preferably by collision-induced dissociation. In the preferred embodiment of the affinity capture tandem mass spectrometer of the present invention, CID is effected in q2 by collision with nitrogen gas at about 10⁻² Torr.

The fragment spectrum is then used to query sequence databases using known algorithms, such as that disclosed in Yates et al., U.S. Pat. Nos. 5,538,897 and 6,017,693, and that employed in Protein Prospector MS-TAG (http://prospector.ucsf/edu) module.

Putative identifications can be further verified by selecting a second parent peptide and repeating the approach, as necessary to confirm that all peptides derive from an identifiable parent.

Thereafter, once the second binding partner is identified, the nature of the intermolecular interaction can be studied as set forth above. The known cleavage rules of the fragmenting enzyme (or chemical, such as CNBr) is applied to the primary sequence of the now-identified second binding partner, and the empirically measured peptides mapped onto the theoretical digest, thus identifying the peptides that had bound to, and thus in the native molecule contribute to the binding to, the immobilized first binding partner. And as above, the experiment can be repeated with increasing stringency of wash to identify those peptides most tightly bound.

Other perturbations can be performed to elucidate further the nature of the intermolecular binding.

The elution characteristics of the eluant to wash the probe following fragmentation of the second binding partner can be altered to identify the fragments that contribute most strongly to the interaction, or to identify pH-dependent or salt-dependent contacts that contribute to binding.

The principle is of course well-known in the chromatographic and molecular biological arts: with increased stringency of wash (e.g., increased salt concentration, higher temperature), those fragments less tightly bound to the immobilized first binding will be eluted off the first binding partner. In the present geometry, such poorly binding fragments will elute off the probe and be lost from the subsequent mass spectrometric analysis. A series of experiments can thus be performed in which the probe, or identical counterpart probes, are washed at increasing stringency, thus creating a graded series of subsets of fragments of the second binding partner, in which each successive subset has a smaller subset of more tightly binding fragments.

As noted above, the first (immobilized) and second (free) binding partners can be interchanged, allowing the other partner's binding contacts to be elucidated.

A further useful perturbation is removal or alteration of post-translational modifications on one or both of the binding partners. For example, if the first binding partner is a glycoprotein, treatment with one or more specific or nonspecific glycosidases prior to, and/or after, binding of the second binding partner will help elucidate the contribution of sugar residues to the binding.

Analogously, where one of the binding partners is nucleic acid, treatment of the nucleic acid binding partner with nuclease after binding of the other binding partner can help identify critical binding residues.

The above-described approach to characterizing intermolecular interactions replaces the multi-platform, labor-intensive, insensitive techniques of the prior art with a single platform, streamlined, sensitive approach. The approach is applicable to a wide variety of different biological systems and problems.

As suggested above, the methods of the present invention can be used to for epitope mapping—that is, to identify the contacts within an antigen that contribute to binding to antibody, T cell receptor, or MHC. The methods can be used to elucidate the nature of binding of biological ligands to their receptors, of transcription factors to nucleic acid, of transcription factors to other transcription factors in a multiprotein complex.

Although particularly discussed above with respect to protein/protein interactions, the methods of the present invention can be practiced to elucidate the binding interactions between lectins and glycoproteins, protein and nucleic acid, and small molecules and receptors.

Particularly with respect to small molecule ligands, the methods can also be applied to the design of agonists and antagonists of known receptors.

Over the past decade, techniques have been developed for combinatorially generating large numbers of small molecules and for screening such molecules in various homogeneous and live cell assays for their ability to affect one or more biological processes. For example, homogeneous scintillation proximity assays can be used to screen combinatorial libraries for binding to a known receptor; digital image-based cellular assays can be used to screen compounds from combinatorial libraries for downstream effects, such as cytoplasmic/nuclear transport of receptors, changes in intracellular calcium distribution, changes in cell motility.

Once such a lead compound is identified, however, a detailed understanding of the interaction of the small molecule with its receptor will facilitate intelligent design of molecules with improved pharmacokinetics and therapeutic index. The techniques of the present invention are well suited for such use.

If the small molecule provides a signal near that provided by the energy absorbing molecules, MS is performed with single ion monitoring looking only for the known mass for the combinatorial library component.

### EXAMPLE 1

### Identification of Prostate Cancer Biomarker

Traditionally, prostatic carcinoma is diagnosed via biopsy after discovery of elevated blood levels of prostate specific antigen (PSA). In normal males, PSA is present at levels of less than 1 ng/ml. For both BPH and prostatic carcinoma, PSA levels may be elevated to 4-10 ng/ml. Chen et al., J. Urology 157:2166-2170 (1997); Qian et al., Clin. Chem. 43:352-359 (1997). PSA is known to have chymotryptic activity, cleaving at the C-terminus of tyrosine and leucine. Qian et al., Clin. Chem. 43:352-359 (1997).

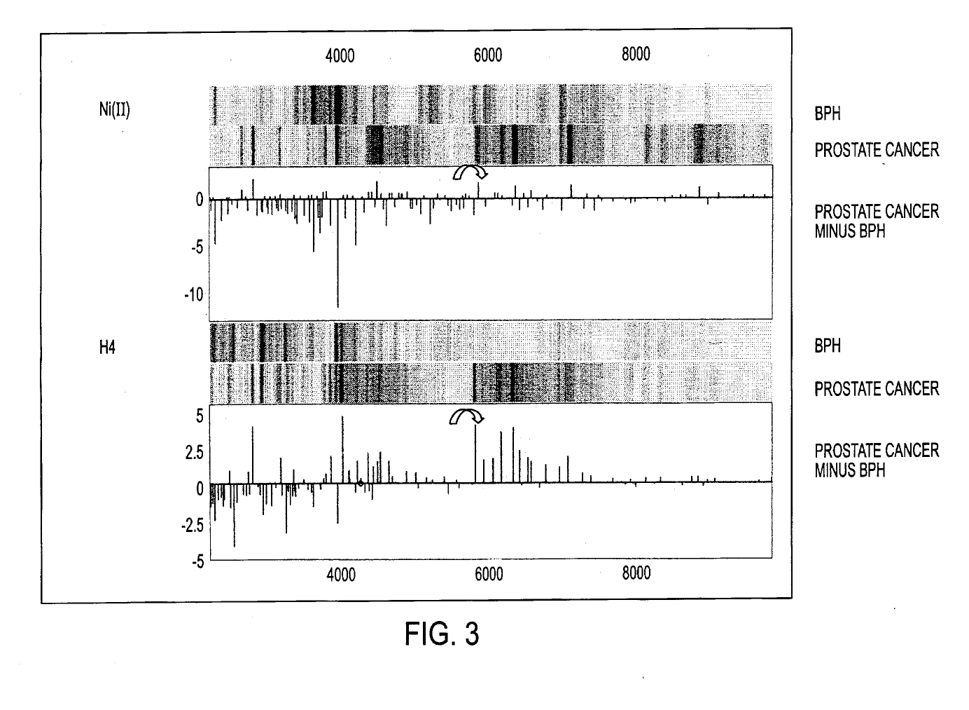

Seminal plasma from patients diagnosed with BPH as well as patients diagnosed with prostatic carcinoma were analyzed using the technique of ProteinChip® differential display. FIG. 3 displays the seminal fluid protein profiles of a single BPH and prostate cancer patient. A virtual gel display is used to enhance visual comparison between samples. A difference plot for the protein profiles of prostate cancer minus BPH is displayed beneath the gel view plots. Positively displaced signals of the difference plot indicate proteins that are upregulated in prostate cancer, while negative peaks represent prostate cancer downward protein regulation. Several uniquely upregulated signals, indicating possible prostate cancer biomarkers, were detected.

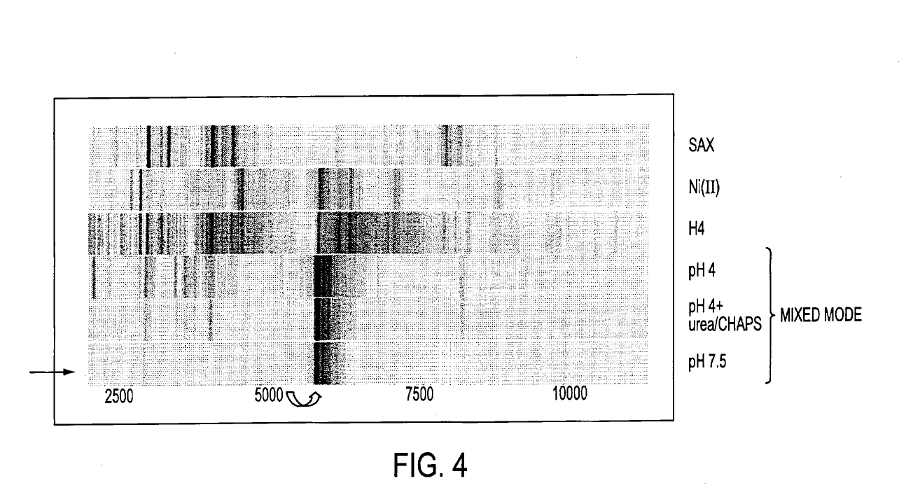

On-chip isolation of one of these upregulated proteins was achieved by using a mixed mode surface and neutral pH buffer wash (see FIG. 4). In this, case, the protein was enriched to near homogeneity. The enriched biomarker candidate was then exposed to in-situ digestion using trypsin. After incubation, a saturated solution of CHCA (matrix) was added and the subsequent digest products analyzed by SELDI-TOF.

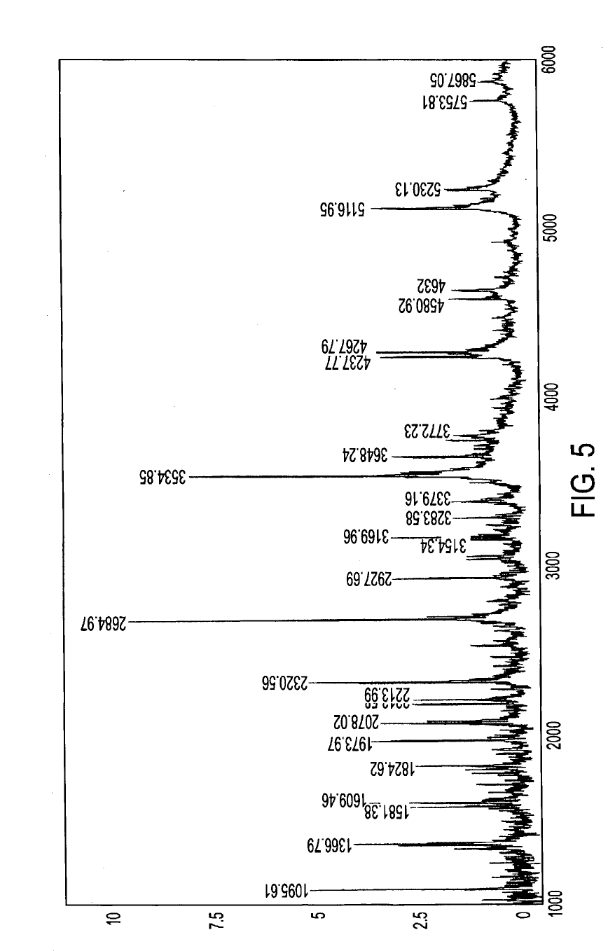

Several peptides were detected (see FIG. 5). The resultant peptide signals were submitted for protein database analysis and a preliminary identification of human semenogellin I was made. This identification was somewhat perplexing, since the biomarker had a molecular weight of about 5751 Da, far less than that of semenogellin I (MW 52,131 Da).

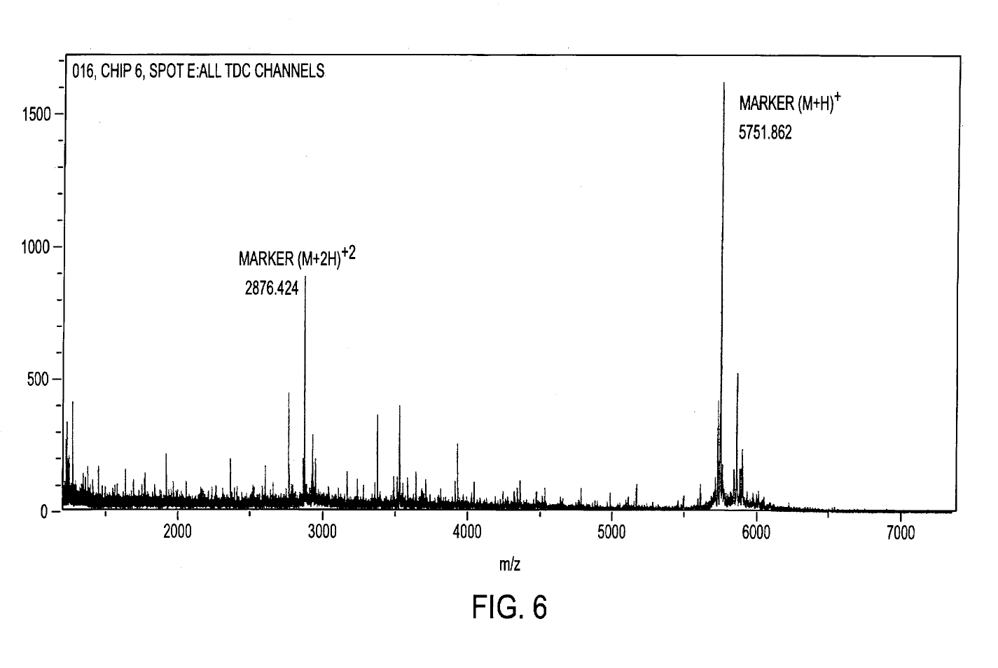

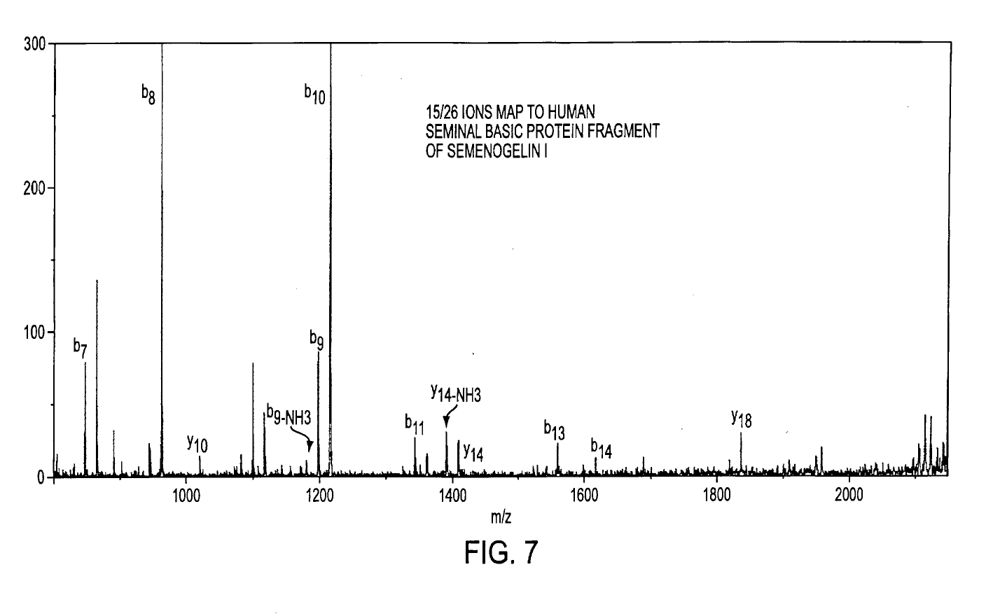

The same purified protein was submitted for ProteinChip LDI Qq-TOF MS detection (see FIG. 6). Because the parent ion at 5751 Da was beyond the current mass limit for LDI Qq-TOF MS/MS analysis (3000 M/z), the doubly charged ion was used for CID MS/MS sequencing (see FIG. 7). The CID MS/MS results were used to perform protein database mining. 15 of the 26 ms/ms ions mapped back to human seminal basic protein (SBP), a proteolytically derived fragment of semenogelin I, providing definitive identification of this candidate biomarker.

While initial studies such as these quickly reveal potential biomarkers, complete validation of any biomarker requires analysis of dozens or even hundreds of relevant samples to obtain statistically significant information regarding expression and prevalence.

All patents, patent publications, and other published references mentioned herein are hereby incorporated by reference in their entireties as if each had been individually and specifically incorporated by reference herein. By their citation of various references in this document, applicants do not admit that any particular reference is “prior art” to their invention.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

What is claimed is:

1. A method of characterizing binding interactions between a first and second molecular binding partner, the method comprising:
binding a second binding partner to a first binding partner, said first binding partner immobilized to a surface of a laser desorption ionization probe;
fragmenting said second binding partner; and then
detecting at least one of said fragments by a tandem mass spectrometer measurement,
the mass spectrum of said detected fragments characterizing said binding interactions.

2. The method of claim 1, further comprising the step, before binding of said second binding partner to said first binding partner, of:
immobilizing a first binding partner to a surface of an affinity capture probe.

3. The method of claim 2, wherein said immobilizing of said first partner to said affinity capture probe surface is a direct binding.

4. The method of claim 3, wherein said direct binding to said probe surface is a covalent bonding.

5. The method of claim 4, wherein said covalent bonding is between an amine of said first binding partner and a carbonyldiimidazole moiety of said probe surface.

6. The method of claim 4, wherein said covalent bonding is between an amino or thiol group of said first binding partner and an epoxy group of said probe surface.

7. The method of claim 3, wherein said direct binding to said probe surface is a noncovalent bonding.

8. The method of claim 7, wherein said direct binding to said probe surface is a coordinate or dative bonding.

9. The method of claim 8, wherein said coordinate or dative bonding is to a metal of said probe surface.

10. The method of claim 9, wherein said metal is selected from the group consisting of gold and platinum.

11. The method of claim 2, wherein said affinity capture probe immobilizing surface is a chromatographic adsorption surface.

12. The method of claim 11, wherein said chromatographic adsorption surface is selected from the group consisting of reverse phase, anion exchange, cation exchange, immobilized metal affinity capture and mixed-mode surfaces.

13. The method of claim 2, wherein said immobilizing of said first partner to said affinity capture probe surface is an indirect binding.

14. The method of claim 13, wherein said indirect binding covalently binds said first binding partner to said affinity capture probe surface.

15. The method of claim 14, wherein said indirect covalent binding to said affinity capture probe surface includes a cleavable linker.

16. The method of claim 15, wherein said linker is cleavable by an agent selected from the group consisting of a chemical, an enzyme, and radiation.

17. The method of claim 13, wherein said indirect binding noncovalently binds said first binding partner to said affinity capture probe surface.

18. The method of claim 17, wherein said noncovalent indirect bonding to said affinity capture probe surface includes a biotin molecule.

19. The method of claim 17, wherein said noncovalent indirect bonding to said affinity capture probe surface includes an avidin molecule.

20. The method of claim 17, wherein said noncovalent indirect bonding to said affinity capture probe surface includes a streptavidin molecule.

21. The method of claim 1, wherein said first molecular binding partner is selected from the group consisting of protein, nucleic acid, carbohydrate, and lipid.

22. The method of claim 21, wherein said first molecular binding partner is a protein.

23. The method of claim 22, wherein said protein is naturally occurring.

24. The method of claim 23, wherein said protein occurs naturally in an organism selected from the group consisting of multicellular eukaryote, single cell eukaryote, prokaryote, and virus.

25. The method of claim 24, wherein said protein occurs naturally in a multicellular eukaryote.

26. The method of claim 25, wherein said multicellular eukaryote is selected from the group consisting of mammals, insects, nematodes, fish, and vascular plants.

27. The method of claim 26, wherein said multicellular eukaryote is a mammal.

28. The method of claim 27, wherein said mammal is homo sapiens.

29. The method of claim 27, wherein said mammal is a rodent.

30. The method of claim 27, wherein said rodent is a mouse, rat, or guinea pig.

31. The method of claim 22, wherein said protein is nonnaturally occurring.

32. The method of claim 31, wherein said protein is a recombinant fusion protein.

33. The method of claim 22, wherein said protein is selected from the group consisting of antibody, receptor, transcription factor, cytoskeletal protein, cell cycle protein, and ribosomal protein.

34. The method of claim 33, wherein said protein is an antibody.

35. The method of claim 33, wherein said protein is a receptor.

36. The method of claim 35, wherein said receptor is selected from the group consisting of cell surface receptor, transmembrane receptor, and nuclear receptor.

37. The method of claim 1, wherein said binding of said second binding partner to said first binding partner is effected by contacting said first binding partner with a biologic sample.

38. The method of claim 37, wherein said biological sample is a fluid selected from the group consisting of blood, lymph, urine, cerebrospinal fluid, synovial fluid, milk, saliva, vitreous humor, aqueous humor, mucus and semen.

39. The method of claim 38, wherein said biological fluid is blood.

40. The method of claim 38, wherein said fluid is urine.

41. The method of claim 38, wherein said biological fluid is CSF.

42. The method of claim 37, wherein said biological sample is a cell lysate.

43. The method of claim 1, wherein said second binding partner is a protein.

44. The method of claim 1, wherein said binding of said second binding partner to said first binding partner is effected by contacting said first binding partner with an aliquot of a chemically synthesized combinatorial library.

45. The method of claim 1, wherein said binding of said second binding partner to said first binding partner is effected by contacting said first binding partner with an aliquot of a biologically displayed combinatorial library.

46. The method of claim 45, wherein said library is a phage-displayed library.

47. The method of claim 1, wherein said fragmenting is effected by contacting said second binding partner with an enzyme.

48. The method of claim 43, wherein said fragmenting is effected by contacting said second binding partner with an enzyme.

49. The method of claim 48, wherein said enzyme is a specific endoprotease.

50. The method of claim 49, wherein said endoprotease is selected from the group consisting of trypsin, Glu-C (V8) protease, endoproteinase Arg-C (serine protease), endoproteinase Arg-C (cysteine protease), Asn-N protease, and Lys-C protease.

51. The method of claim 50, wherein said protease is trypsin.

52. The method of claim 1, wherein said fragmenting is effected by contacting said second binding partner with a liquid phase chemical.

53. The method of claim 52, wherein said chemical is CNBr.

54. The method of claim 1, further comprising the step, after binding of said second binding partner to said first binding partner, and before fragmenting said second binding partner, of:
denaturing said second binding partner.

55. The method of claim 1, further comprising the step, after fragmenting said second binding partner, of:
washing said probe with a first eluant.

56. The method of claim 55, further comprising, after washing said probe with a first eluant and before detecting fragments by a tandem mass spectrometer measurement, the step of:
washing said probe with a second eluant, said second eluant differing from said first eluant in at least one elution characteristic.

57. The method of claim 56, wherein said elution characteristic is selected from the group consisting of pH, ionic strength, detergent strength, and hydrophobicity.

58. The method of claim 1 or 55, further comprising the step, after fragmenting and before detecting said fragments, of:
applying energy absorbing molecules to said probe.

59. The method of claim 1, further comprising the steps, after fragmenting and before detecting said fragments, of:
engaging said probe in the affinity capture probe interface of an analytical instrument, said analytical instrument comprising:
a laser desorption ionization source;
an affinity capture probe interface; and
a tandem mass spectrometer,
wherein said affinity capture probe interface is capable of engaging an affinity capture probe and positioning said probe in interrogatable relationship to said laser source and concurrently in communication with said tandem mass spectrometer; and then
desorbing and ionizing fragments of said second binding partner from said probe using said laser source.

60. The method of claim 1, wherein said tandem mass spectrometer measurement is a measurement of all ion masses.

61. The method of claim 1, wherein said tandem mass spectrometer measurement is a measurement of masses of a subset of fragments.

62. The method of claim 1, wherein said tandem mass spectrometer measurement is a single ion monitoring measurement.

63. The method of claim 1, further comprising the step, after said detecting, of
comparing said fragment measurements with those predicted by applying cleavage rules of the fragmenting enzyme to the primary amino acid sequence of said second binding partner.

64. The method of claim 63, further comprising the step, after said detecting and before said comparing, of
identifying said second binding partner through ms/ms analysis.

65. The method of claim 64, wherein said identifying through ms/ms analysis comprises the steps of:
mass spectrometrically selecting a first fragment of said second binding partner;
dissociating said second binding partner first fragment in the gas phase;
measuring the fragment spectrum of said second binding partner first fragment, and then
comparing said fragment spectrum to fragment spectra predicted from amino acid sequence data prior-accessioned in a database.

66. The method of claim 65, wherein said amino acid sequence data are selected from the group consisting of empiric and predicted data.

67. The method of claim 65, wherein said dissociating is collision induced dissociation.

68. The method of claim 1, wherein said first binding partner is an antibody.

69. The method of claim 1, wherein said first binding partner is a T cell receptor.

70. The method of claim 1, wherein said first binding partner is an MHC molecule.

71. The method of claim 1, wherein said first binding partner is a receptor and said second binding partner is an agonist of said receptor.

72. The method of claim 1, wherein said first binding partner is a receptor and said second binding partner is a partial agonist of said receptor.

73. The method of claim 1, wherein said first binding partner is a receptor and said second binding partner is an antagonist of said receptor.

74. The method of claim 1, wherein said first binding partner is a receptor and said second binding partner is a partial antagonist of said receptor.

75. The method of claim 1, wherein said first binding partner is a glycoprotein receptor and said second binding partner is a lectin.
